(19) United States
(12) Patent Application Publication
(10) Pub. No.: US 2003/0233022 A1
(43) Pub. Date: Dec. 18, 2003

(54) Devices and methods for heart valve treatment

(76) Inventors: Robert M. Vidlund, Maplewood, MN (US); Cyril J. Schweich JR., Maple Grove, MN (US); Todd J. Mortier, Minneapolis, MN (US); Craig A. Ekvall, Elk River, MN (US); Jason E. Kalgreen, Plymouth, MN (US); Richard Schroeder, Fridley, MN (US); Jeffrey P. LaPlante, Minneapolis, MN (US)

Correspondence Address: Susanne T. Jones, FINNEGAN, HENDERSON, FARABOW,, GARRETT & DUNNER, L.L.P., 1300 I Street, N.W., Washington, DC, 20005-3315, US

(21) Appl. No.: 10/456,751

(22) Filed: Jun. 9, 2003

Related U.S. Application Data
(60) Provisional application No. 60/387,558, filed on Jun. 12, 2002.

(51) Int. Cl. A61N 1/362

(52) U.S. Cl. 600/16

(57) ABSTRACT

Devices, systems and methods for improving the function of a valve of a heart by implanting the device adjacent the valve such that the device indirectly applies a force to the valve and increases coaptation of the leaflets, or otherwise improves valve function. The device may be implanted in a position that does not directly contact the valve structures. The force may be applied to a wall of the heart, and the force may be an inward force applied to two walls of the heart, such as the left ventricular free wall and the ventricular septum, or the left ventricular free wall and the right ventricular free wall, to improve mitral valve function.

### RELATED APPLICATIONS

This patent application claims the benefits of priority of U.S. Provisional Application No. 60/387,558, filed Jun. 12, 2002, the entire contents of which are incorporated herein by reference.

U.S. patent application Ser. No. 09/680,435, filed Oct. 6, 2000, entitled METHODS AND DEVICES FOR IMPROVING MITRAL VALVE FUNCTION (hereinafter referred to as the “435 patent application”), and U.S. patent application Ser. No. 10/040,784, filed Jan. 9, 2002, entitled DEVICES AND METHODS FOR HEART VALVE TREATMENT (hereinafter referred to as the “784 patent application”) also are incorporated herein by reference in their entirety.

### FIELD OF THE INVENTION

The present invention relates to devices and associated methods for treating and improving the performance of dysfunctional heart valves. More particularly, the invention relates to devices and methods that passively assist to reshape a dysfunctional heart valve to improve its performance.

### BACKGROUND OF THE INVENTION

Various etiologies may result in heart valve insufficiency depending upon both the particular valve as well as the underlying disease state of the patient. For instance, a congenital defect may be present resulting in poor coaptation of the valve leaflets, such as in the case of a monocusp aortic valve, for example. Valve insufficiency also may result from an infection, such as rheumatic fever, for example, which may cause a degradation of the valve leaflets. Functional regurgitation also may be present. In such cases, the valve components may be normal pathologically, yet may be unable to function properly due to changes in the surrounding environment. Examples of such changes include geometric alterations of one or more heart chambers and/or decreases in myocardial contractility. In any case, the resultant volume overload that exists as a result of an insufficient valve may increase chamber wall stress. Such an increase in stress may eventually result in a dilatory process that further exacerbates valve dysfunction and degrades cardiac efficiency.

Mitral valve regurgitation often may be driven by the functional changes described above. Alterations in the geometric relationship between valvular components may occur for numerous reasons, including events ranging from focal myocardial infarction to global ischemia of the myocardial tissue. Idiopathic dilated cardiomyopathy also may drive the evolution of functional mitral regurgitation. These disease states often lead to dilatation of the left ventricle. Such dilatation may cause papillary muscle displacement and/or dilatation of the valve annulus. As the papillary muscles move away from the valve annulus, the chordae connecting the muscles to the leaflets may become tethered. Such tethering may restrict the leaflets from closing together, either symmetrically or asymmetrically, depending on the relative degree of displacement between the papillary muscles. Moreover, as the annulus dilates in response to chamber enlargement and increased wall stress, increases in annular area and changes in annular shape may increase the degree of valve insufficiency. Annular dilatation is typically concentrated on the posterior aspect, since this aspect is directly associated with the dilating left ventricular free wall and not directly attached to the fibrous skeleton of the heart. Annular dilatation also may result in a flattening of the valve annulus from its normal saddle shape.

Alterations in functional capacity also may cause valve insufficiency. In a normally functioning heart, the mitral valve annulus contracts during systole to assist in leaflet coaptation. Reductions in annular contractility commonly observed in ischemic or idiopathic cardiomyopathy patients therefore hamper the closure of the valve. Further, in a normal heart, the papillary muscles contract during the heart cycle to assist in maintaining proper valve function. Reductions in or failure of the papillary muscle function also may contribute to valve regurgitation. This may be caused by infarction at or near the papillary muscle, ischemia, or other causes, such as idiopathic dilated cardiomyopathy, for example.

The degree of valve regurgitation may vary, especially in the case of functional insufficiency. In earlier stages of the disease, the valve may be able to compensate for geometric and/or functional changes in a resting state. However, under higher loading resulting from an increase in output requirement, the valve may become incompetent. Such incompetence may only appear during intense exercise, or alternatively may be induced by far less of an exertion, such as walking up a flight of stairs, for example.

Conventional techniques for managing mitral valve dysfunction include either surgical repair or replacement of the valve or medical management of the patient. Medical management typically applies only to early stages of mitral valve dysfunction, during which levels of regurgitation are relatively low. Such medical management tends to focus on volume reductions, such as diuresis, for example, or afterload reducers, such as vasodilators, for example.

Early attempts to surgically treat mitral valve dysfunction focused on replacement technologies. In many of these cases, the importance of preserving the native subvalvular apparatus was not fully appreciated and many patients often acquired ventricular dysfunction or failure following the surgery. Though later experience was more successful, significant limitations to valve replacement still exist. For instance, in the case of mechanical prostheses, lifelong therapy with powerful anticoagulants may be required to mitigate the thromboembolic potential of these devices. In the case of biologically derived devices, in particular those used as mitral valve replacements, the long-term durability may be limited. Mineralization induced valve failure is common within ten years, even in younger patients. Thus, the use of such devices in younger patient groups is impractical.

Another commonly employed repair technique involves the use of annuloplasty rings. These rings originally were used to stabilize a complex valve repair. Now, they are more often used alone to improve mitral valve function. An annuloplasty ring has a diameter that is less than the diameter of the enlarged valve annulus. The ring is placed in the valve annulus and the tissue of the annulus sewn or otherwise secured to the ring. This causes a reduction in the annular circumference and an increase in the leaflet coaptation area. Such rings, however, generally flatten the natural saddle shape of the valve and hinder the natural contractility of the valve annulus. This may be true even when the rings have relatively high flexibility.

To further reduce the limitations of the therapies described above, purely surgical techniques for treating valve dysfunction have evolved. Among these surgical techniques is the Alfiere stitch or so-called bowtie repair. In this surgery, a suture is placed substantially centrally across the valve orifice between the posterior and anterior leaflets to create leaflet apposition. Another surgical technique includes plication of the posterior annular space to reduce the cross-sectional area of the valve annulus. A limitation of each of these techniques is that they typically require opening the heart to gain direct access to the valve and the valve annulus. This generally necessitates the use of cardiopulmonary bypass, which may introduce additional morbidity and mortality to the surgical procedures. Additionally, for each of these procedures, it is very difficult, if not impossible, to evaluate the efficacy of the repair prior to the conclusion of the operation.

Due to these drawbacks, devising effective techniques that could improve valve function without the need for cardiopulmonary bypass and without requiring major remodeling of the valve may be advantageous. In particular, passive techniques to change the shape of the heart chamber and/or associated valve and reduce regurgitation while maintaining substantially normal leaflet motion may be desirable. Further, advantages may be obtained by a technique that reduces the overall time a patient is in surgery and under the influence of anesthesia. It also may be desirable to provide a technique for treating valve insufficiency that reduces the risk of bleeding associated with anticoagulation requirements of cardiopulmonary bypass. In addition, a technique that can be employed on a beating heart would allow the practitioner an opportunity to assess the efficacy of the treatment and potentially address any inadequacies without the need for additional bypass support

### SUMMARY OF THE INVENTION

To address one or more of these unmet needs, an aspect of the present invention, as embodied and broadly described herein, includes a device, system and method for improving the function of a valve of a heart by implanting the device adjacent the valve such that the device indirectly applies a force to the valve and increases coaptation of the leaflets, or otherwise improves valve function. The device may be implanted in a position that does not directly contact the valve structures, including the leaflets, chordae, annulus, and/or papillary muscles. The force may be applied to a wall of the heart, such as the left ventricular free wall, for example, to affect the function of the mitral valve. The indirect force may be an inward force, and the force may be applied to two walls of the heart, such as the left ventricular free wall and the ventricular septum, or the left ventricular free wall and the right ventricular free wall, for example.

The force may be applied with a device that includes an elongate member with one or more anchors attached to the ends thereof. The elongate member may extend through a chamber of the heart, and the anchors may be disposed on an exterior heart wall and/or an interior heart wall.

According to another exemplary aspect of the invention, a device for improving the function of a heart comprises an elongate member configured to be positioned transverse a chamber of the heart and a release mechanism fixedly connected to the elongate member. The release mechanism may be configured to releasably engage with each of a plurality of anchoring members having differing configurations to releasably attach the elongate member to each of the plurality of anchoring members one at a time.

Yet another exemplary aspect includes a method for improving the function of a heart comprising providing a plurality of anchoring members having differing configurations and an elongate member with a release mechanism connected to the elongate member, the release mechanism being configured to releasably engage with each of the a plurality of anchoring members. The method further comprises selecting one of the plurality of anchoring members, positioning the elongate member transverse a chamber of the heart, and engaging the release mechanism with the selected anchoring member so as to releasably attach the elongate member to the selected anchoring member.

According to yet another exemplary aspect, the invention may include a method of delivering a device to be positioned relative to a heart chamber comprising providing an elongate member having a first end and a second end, the second end having an expandable anchoring member attached thereto. The method may further include advancing the first end of the elongate member through a first heart wall, a septal wall, and a second heart wall substantially opposite the septal wall such that the elongate member extends substantially transverse a heart chamber and expanding the expandable anchoring member such that the expandable anchoring member prevents the second end of the elongate member from being able to pass through the septal wall and into the heart chamber.

Yet another exemplary aspect of the invention includes a device for securing an elongate member in a position transverse at least one heart chamber which comprises an anchor assembly configured to be secured to the elongate member. The anchor assembly has a collapsed configuration and an expanded configuration and comprises a first arm, a second arm, and at least one biasing member connecting the first arm and the second arm, wherein, in the absence of external force, the biasing member is configured to exert a biasing force on the first arm and the second arm such that the anchor assembly is in the expanded configuration.

Another exemplary aspect of the invention includes an alignment device comprising an arm and a tissue engaging member configured to engage a tissue surface connected to the arm. The tissue engaging member comprises a cover defining a cover opening, and a rotatable insert defining a plurality of openings configured to be individually aligned with the cover opening by rotating the insert with respect to the cover. When the cover opening and one of the plurality of openings are aligned, the cover opening and one of the plurality of openings are configured to receive a needle assembly.

It should be understood that the invention could be practiced without performing one or more of the preferred objects and/or advantages described above. Other objects of the invention will become apparent from the detailed description which follows.

### BRIEF DESCRIPTION OF THE DRAWINGS

Aside from the structural and procedural arrangements set forth above, the invention could include a number of other arrangements, such as those explained hereinafter. It is to be understood that both the foregoing and the following descriptions are exemplary. The accompanying drawings are included to provide a further understanding of the invention and are incorporated in and constitute a part of this specification. The drawings illustrate exemplary embodiments of the invention and, together with the description, serve to explain certain principles.

FIG. 1A is a superior, short axis, cross-sectional view of a human heart during diastole, showing a mitral valve splint extending through the heart and aligned generally orthogonal to the arcuate opening of the mitral valve;

FIG. 1B is a lateral, long axis, cross-sectional view of the human heart and an exemplary embodiment of mitral valve splint of FIG. 1A;

FIG. 1C is an anterior, long axis view of the human heart and an exemplary embodiment of a mitral valve splint of FIG. 1A;

FIG. 2A is a superior, short axis, cross-sectional view of a human heart showing an incompetent mitral valve during systole;

FIG. 2B is a superior, short axis, cross-sectional view of the human heart of FIG. 2A showing the formerly incompetent mitral valve during systole corrected with an exemplary embodiment of a mitral valve splint;

FIGS. 3A-3C are side and perspective views of an exemplary embodiment of an anterior pad for use with the mitral valve splint shown in FIG. 1;

FIGS. 4A-4G are side and perspective views of an exemplary embodiment of a posterior pad for use with the mitral valve splint shown in FIG. 1;

FIG. 5A is a perspective view of an exemplary embodiment of a mitral valve splint delivery system including a positioning and alignment device (shown in the closed position) and a needle delivery assembly;

FIG. 5B is a perspective view of a portion of the delivery system of FIG. 5A, shown in the open position;

FIG. 5C is a schematic illustration of exemplary embodiments of the needle delivery assembly;

FIGS. 5D and 5E are perspective views of the anterior and posterior vacuum chambers, respectively, of the positioning and alignment device shown in FIG. 5A;

FIGS. 5F and 5G are exploded views of the anterior and posterior vacuum chambers, of FIGS. 5D and 5E, respectively;

FIG. 5H is a perspective view of an exemplary embodiment of a rotating insert for use in the posterior vacuum chamber of the mitral valve delivery system shown in FIG. 5A;

FIG. 5I is a perspective view of a capture plate for use in the posterior vacuum chamber of the mitral valve delivery system shown in FIG. 5A;

FIG. 5J is a schematic plan view of the delivery system of FIG. 5A with the positioning and alignment device disposed on the heart and the needle delivery assembly fully inserted through the heart;

FIGS. 6A-6D are schematic illustrations of an exemplary embodiment of a septal delivery system and method for a mitral valve splint;

FIGS. 7A-7E are schematic illustrations of an exemplary embodiment of an alternative septal delivery system and method for a mitral valve splint;

FIGS. 8A-8F are schematic illustrations of an exemplary embodiment of an endovascular septal delivery system and method for a mitral valve splint;

FIGS. 9A-9D are perspective views of an exemplary embodiment of an expandable pad and associated components for use with the mitral valve splints of FIGS. 6-8; and

FIGS. 10A-10C are schematic views of an exemplary embodiment of an alternative expandable pad for use with the septal mitral valve splints of FIGS. 6-8.

### DETAILED DESCRIPTION OF EXEMPLARY EMBODIMENTS

The various aspects of the devices and methods described herein generally pertain to devices and methods for treating heart conditions, including, for example, dilatation, valve incompetencies, including mitral valve leakage, and other similar heart failure conditions. Each disclosed device may operate passively in that, once placed in the heart, it does not require an active stimulus, either mechanical, electrical, or otherwise, to function. Implanting one or more of the devices operates to assist in the apposition of heart valve leaflets to improve valve function.

In addition, these devices may either be placed in conjunction with other devices that, or may themselves function to, alter the shape or geometry of the heart, locally and/or globally, and thereby further increase the heart's efficiency. That is, the heart experiences an increased pumping efficiency through an alteration in its shape or geometry and concomitant reduction in stress on the heart walls, and through an improvement in valve function.

However, the devices disclosed herein for improving valve function can be “stand-alone” devices, that is, they do not necessarily have to be used in conjunction with additional devices for changing the shape of a heart chamber or otherwise reducing heart wall stress. It also is contemplated that a device for improving valve function may be placed relative to the heart without altering the shape of the chamber, and only altering the shape of the valve itself.

The devices and methods described herein offer numerous advantages over the existing treatments for various heart conditions, including valve incompetencies. The devices are relatively easy to manufacture and use, and the surgical techniques and tools for implanting the devices do not require the invasive procedures of current surgical techniques. For instance, the surgical technique does not require removing portions of the heart tissue, nor does it necessarily require opening the heart chamber or stopping the heart during operation. For these reasons, the surgical techniques for implanting the devices disclosed herein also are less risky to the patient than other techniques. The less invasive nature of these surgical techniques and tools may also allow for earlier intervention in patients with heart failure and/or valve incompetencies.

The devices and methods described herein involve geometric reshaping of the heart and treating valve incompetencies. In certain aspects of the devices and methods described herein, substantially the entire chamber geometry is altered to return the heart to a more normal state of stress. Models of this geometric reshaping, which includes a reduction in radius of curvature of the chamber walls with ventricular splints, may be found in U.S. Pat. Nos. 5,961,440 and 6,050,936, the entire disclosures of these patents are inorporated herein by reference. Prior to reshaping the chamber geometry, the heart walls experience high stress due to a combination of both the relatively large increased diameter of the chamber and the thinning of the chamber wall. Filling pressures and systolic pressures are typically high as well, further increasing wall stress. Geometric reshaping reduces the stress in the walls of the heart chamber to increase the heart's pumping efficiency, as well as to stop further dilatation of the heart.

Although the methods and devices are discussed hereinafter in connection with their use in the left ventricle and for the mitral valve of the heart, these methods and devices may be used in other chambers and for other valves of the heart for similar purposes. One of ordinary skill in the art would understand that the use of the devices and methods described herein also could be employed in other chambers and for other valves of the heart. The left ventricle and the mitral valve have been selected for illustrative purposes because a large number of the disorders occur in the left ventricle and in connection with the mitral valve.

The following detailed description of exemplary embodiments of the present invention is made with reference to the drawings, in which similar elements in different drawings are numbered the same. The drawings, which are not necessarily to scale, depict illustrative embodiments and are not intended to limit the scope of the invention.

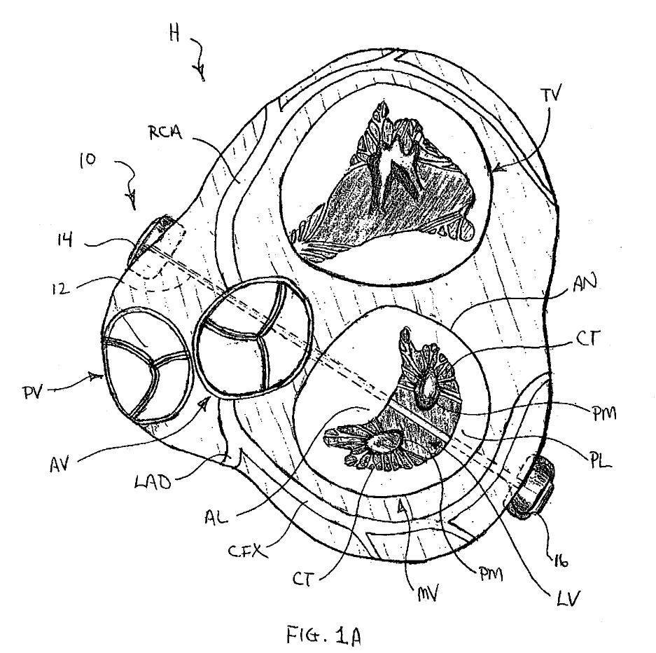

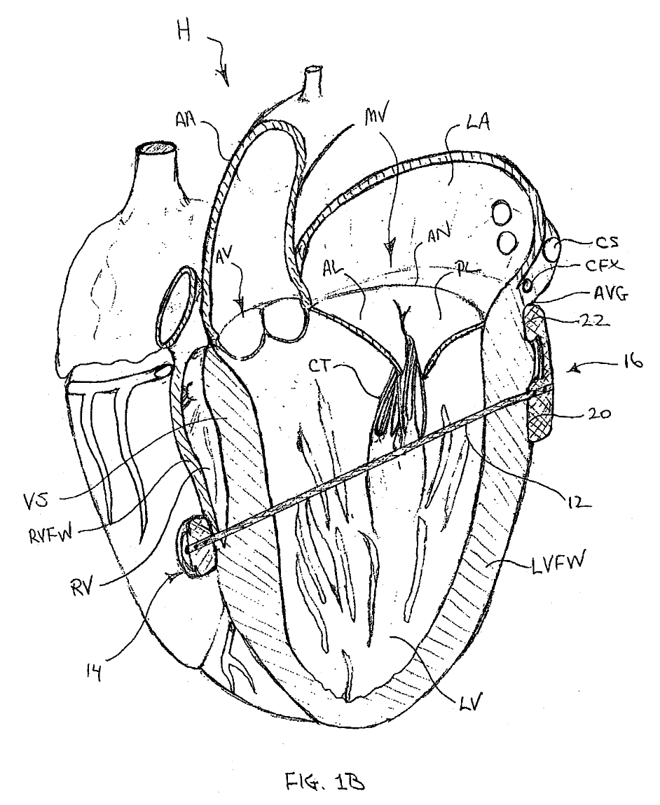

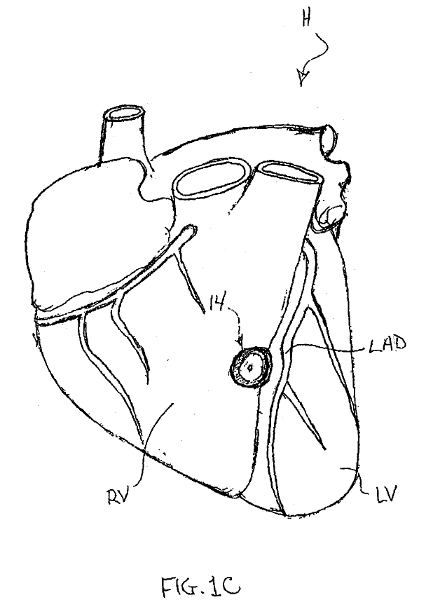

With reference to FIGS. 1A, 1B and 1C, a human heart H is shown during diastole. The devices and methods described herein are discussed with reference to the human heart H, but may also be applied to other animal hearts not specifically mentioned herein. A superior, short axis, cross-sectional view of the heart H is shown in FIG. 1A, a lateral, long axis, cross-sectional view of the human heart H is shown in FIG. 1B, and an anterior, long axis view of the human heart H is shown in FIG. 1C. In FIGS. 1A-1C, a mitral valve splint 10 is shown, which generally includes an elongate tension member 12 secured to an anterior pad 14 and a posterior pad 16.

For purposes of discussion and illustration, several anatomical features of the human heart are labeled as follows: left ventricle LV; right ventricle RV; left atrium LA; ventricular septum VS; right ventricular free wall RVFW; left ventricular free wall LVFW; atrioventricular groove AVG; mitral valve MV; tricuspid valve TV; aortic valve AV; pulmonary valve PV; papillary muscle PM; chordae tendeneae CT (or simply chordae); anterior leaflet AL; posterior leaflet PL; annulus AN; ascending aorta AA; coronary sinus CS; right coronary artery RCA; left anterior descending artery LAD; and circumflex artery CFX.

FIGS. 1A and 1B illustrate the mitral valve splint 10 extending through the heart H. As seen in FIG. 1A, the splint 10 substantially bisects the projection of the opening of the mitral valve MV and is aligned generally orthogonal to the arcuate opening defined between the anterior leaflet AL and posterior leaflet PL of the mitral valve MV. As seen in FIG. 1B, the splint 10 extends across the left ventricle LV at an inferior angle from the superior aspect of the left ventricular free wall LVFW, through the ventricular septum VS, and across the right ventricle RV near the intersection of the right ventricle RV and ventricular septum VS.

Both the anterior pad 14 and the posterior pad 16 are seated on the epicardium, while the tension member 12 extends through the myocardium and the ventricular chamber(s). This position also allows for the mitral valve splint 10 to have both pads 14, 16 placed epicardially, avoiding the need to position a pad interior to any of the heart chambers. To avoid interference with mitral valve MV function, the pads 14, 16 may be positioned such that the tension member 12 extends inferiorly of the of the leaflets AL/PL and chordae CT of the mitral valve MV. To maximize shape change effects of the mitral valve MV, and in particular the papillary muscles PM and/or annulus AN, the posterior pad 16 may have an inferior contact zone 20 and a superior contact zone 22, positioned on the epicardial surface proximate the papillary muscles PM and annulus AN, respectively.

The posterior pad 16 may be positioned such that the superior contact zone 22 rests in, or proximate to, the atrioventricular groove AVG, which is adjacent the annulus AN of the mitral valve MV. In this position, the application of deforming forces brought about by the posterior pad 16 causes a direct deformation of the annulus AN of the mitral valve MV, and/or repositioning of the papillary muscles PM. Both of these actions contribute to better coaptation of the leaflets AL, PL, minimizing or eliminating mitral valve regurgitation.

The anterior pad 14 may be positioned on the epicardial surface of the right ventricle RV, proximate the base of the right ventricular outflow track, and close to the intersection of the right ventricular free wall RVFW and the interventricular septum VS. In this position, the function of the right ventricle is minimally impacted when the splint 10 is tightened. Also in this position, the anterior pad 14 avoids interference with important blood vessels as well as important conduction pathways. For example, as seen in FIG. 1C, the anterior pad 14 may be so positioned to one side of the left anterior descending coronary artery LAD to avoid interference therewith.

The position of the splint 10 as shown in FIGS. 1A and 1B is exemplary, and it is anticipated that the position of the splint 10 may be virtually any orientation relative to the mitral valve MV leaflets AL, PL, depending on the heart failure and mitral valve regurgitation associated with the particular heart at issue. It is also contemplated that the mitral valve splint 10 may be utilized in conjunction with additional ventricular shape change devices such as those described in U.S. Pat. No. 6,261,222 to Schweich, Jr., et al., and/or U.S. Pat. No. 6,183,411 to Mortier, et al., the entire disclosures of which are incorporated herein by reference.

The mitral valve splint 10 may improve mitral valve function through a combination of effects. First, the shape of the annulus AN is directly altered, preferably during the entire cardiac cycle, thereby reducing the annular cross sectional area and bringing the posterior leaflet PL in closer apposition to the anterior leaflet AL. Second, the position and rotational configuration of the papillary muscles PM and surrounding areas of the left ventricle LV are further altered by the tightening of the splint 10. This places the chordae CT in a more favorable state of tension, allowing the leaflets AL, PL to more fully appose each other. Third, since the annulus AN of the mitral valve MV is muscular and actively contracts during systole, changing the shape of the annulus AN will also reduce the radius of curvature of at least portions of the annulus AN, just as the shape change induced by ventricular splints discussed hereinbefore reduces the radius of at least significant portions of the ventricle. This shape change and radius reduction of the annulus AN causes off-loading of some of the wall stress on the annulus AN. This, in turn, assists the annulus's ability to contract to a smaller size, thereby facilitating full closure of the mitral valve MV during systole.

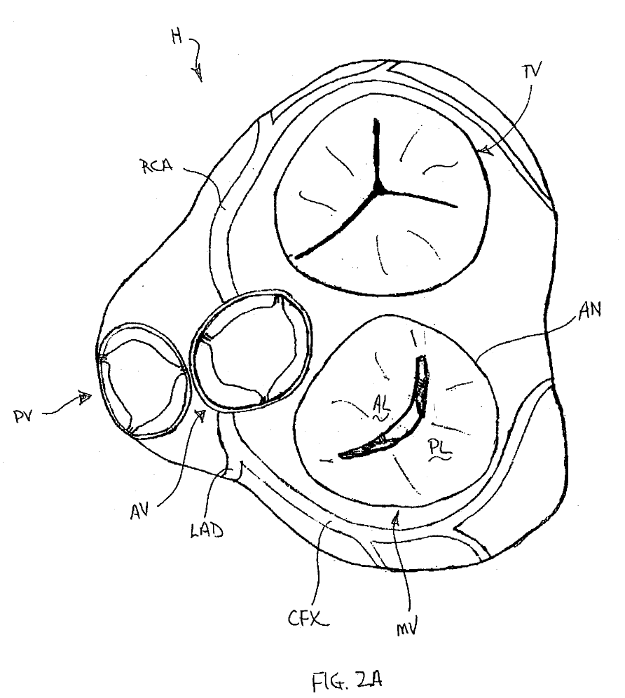

These effects are illustrated in FIGS. 2A and 2B. FIG. 2A shows an incompetent mitral valve MV during systole. The mitral valve MV is rendered incompetent by, for example, a dilated valve annulus AN. The mitral valve MV may become incompetent by several different mechanisms including, for example, a dilated valve annulus AN as mentioned above, or a displaced papillary muscle PM due to ventricular dilation. FIG. 2B shows the formerly incompetent mitral valve MV of FIG. 2A during systole as corrected with a mitral valve splint 10. As seen in FIG. 2B, the splint 10 causes inward displacement of a specific portion of the left ventricular free wall LVFW, resulting in a re-configuration and re-shaping of the annulus AN and/or the papillary muscles PM, thus providing more complete closure of the mitral valve leaflets AL, PL during systole.

As mentioned hereinbefore, the mitral valve splint 10 generally includes an elongate tension member 12 secured to an anterior pad or anchor 14 and a posterior pad or anchor 16. The pads 14, 16 may essentially function as epicardial anchors that engage the heart wall, do not penetrate the heart wall, and provide surfaces adjacent the exterior of the heart wall to which the tension member 12 is connected.

Tension member 12 may comprise a composite structure including an inner cable to provide mechanical integrity and an outer covering to provide biocompatibility. By way of example, not limitation, the inner cable of tension member 12 may have a braided-cable construction such as a multifilar braided polymeric construction. In general, the filaments forming the inner cable of the tension member 12 may comprise high performance fibers. For example, the inner cable may comprise filaments of ultra high molecular weight polyethylene available under the trade names Spectra™ and Dyneema™, or the inner cable may comprise filaments of some other suitable material such as polyester available under the trade name Dacron™ or liquid crystal polymer available under the trade name Vectran™.

The filaments forming the inner cable may be combined in yarn bundles of approximately 50 individual filaments, with each yarn bundle being approximately 180 denier. For example, two bundles may be paired together (referred to as 2-ply) and then braided with approximately 16 total bundle pairs to form the inner cable. The braided cable may include, for example, approximately 20 to 50 picks per inch (number of linear yarn overlaps per inch), such as approximately 30 picks per inch. The inner cable may have an average diameter of approximately 0.030 to 0.080 inches, for example, or approximately 0.055 inches, with approximately 1600 individual filaments. Further aspects of the inner cable of the tension member 12 are described in pending U.S. patent application Ser. No. 09/532,049, filed Mar. 21, 2000, entitled A SPLINT ASSEMBLY FOR IMPROVING CARDIAC FUNCTION IN HEARTS, AND METHOD FOR IMPLANTING THE SPLINT ASSEMBLY (hereinafter referred to as the “049 patent application”), the entire disclosure of which is incorporated herein by reference.

When formed within the parameters indicated above, the inner cable permits the tension member 12 to withstand the cyclical stresses occurring within the heart chamber without breaking or weakening; provides a strong connection to the pads 14, 16; minimizes damage to internal vascular structure and the heart tissue; and minimizes the obstruction of blood flow within the heart chamber. Although exemplary parameters for the inner cable of the tension member 12 have been described above, it is contemplated that other combinations of material, yarn density, number of bundles, and pick count may be used, so as to achieve one or all the desired characteristics noted above.

The outer covering surrounding the inner cable of the tension member 12 may provide properties that facilitate sustained implantation in the heart. In particular, because tension member 12 may be in blood contact as it resides within a chamber of the heart H, the outer covering provides resistance to thrombus generation. Furthermore, because of the relative motion that occurs between the heart H and certain portions of tension member 12 passing through the heart chamber walls, the covering allows for tissue ingrowth to establish a relatively firm bond between the tension member 12 and the heart wall, thus reducing relative motion therebetween and minimizing potential irritation of the heart wall.

The outer covering surrounding the inner cable of the tension member 12 may be made of a porous expanded polytetrafluoroethylene (ePTFE) sleeve. The ePTFE material is biostable and tends not to degrade or corrode in the body. The ePTFE sleeve may have an inner diameter of approximately 0.040 inches and a wall thickness of approximately 0.005 inches, for example, prior to placement around the inner cable of the tension member 12. The inner diameter of covering may stretch to fit around the inner cable to provide a frictional fit therebetween. The ePTFE material of the covering may have an internodal distance of between approximately 20 and approximately 70 microns, such as approximately 45 microns, for example. This may permit cellular infiltration and thus result in secure ingrowth of the adjacent heart wall tissue so as to create a tissue surface on the tension member 12 residing in the heart chamber. The ePTFE material, particularly having the internodal spacing discussed above, has a high resistance to thrombus formation and withstands the cyclic bending environment occurring in the heart. Further aspects of the outer covering of the tension member 12 are described in the '049 patent application. Although ePTFE has been described as a suitable material for the outer covering of the tension member 12, other suitable materials exhibiting similar characteristics may also be used.

The anterior pad 14 and the posterior pad 16 of the mitral valve splint 10 are connected to opposite ends of the tension member 12. To facilitate delivery of the splint 10 as described in more detail hereinafter, one of the anchor pads 14, 16 may be fixed and locked to the tension member 12 prior to implantation. The other of the anchor pads 14, 16 may be initially adjustable and subsequently fixed to the tension member 12. In particular, its position along the length of the tension member 12 may be adjusted during implantation, prior to fixation to the tension member 12. The posterior pad 16 may be positioned proximate the posterior leaflet PL of the mitral valve MV and may be fixed relative to tension member 12. The anterior pad 14 may be positioned near the intersection of the right ventricle RV and ventricular septum VS, and may be initially adjustable relative to tension member 12 and subsequently fixed thereto.

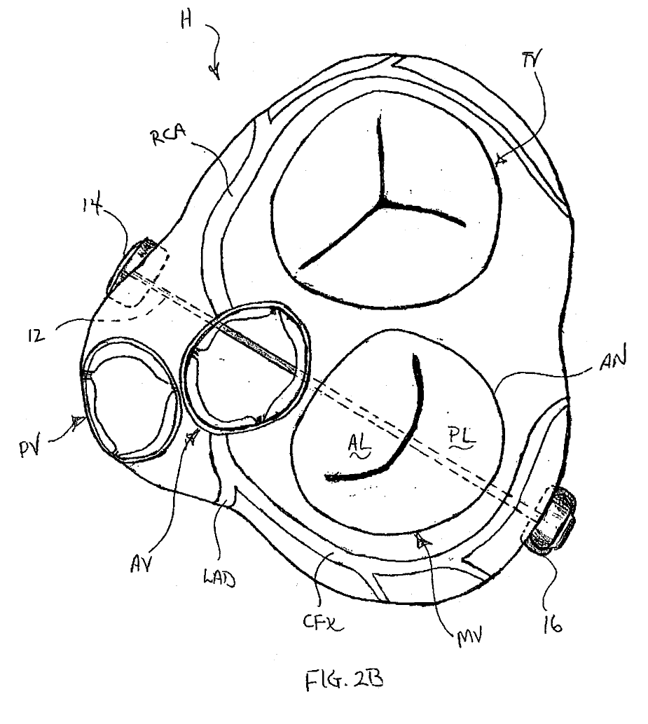

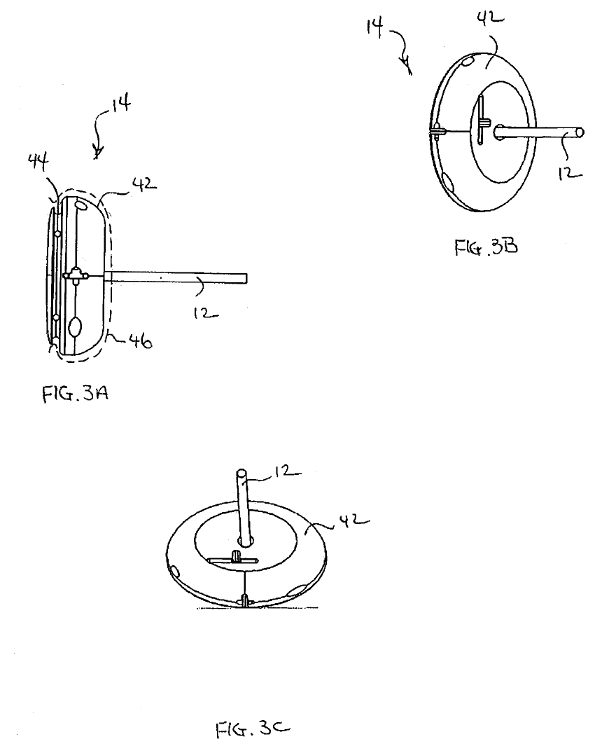

In the exemplary embodiments described herein, the anterior pad 14 is an adjustable pad, but may be fixed as well. The anterior pad 14 may have a substantially circular shape as shown in FIG. 1C or an oval shape as shown in FIGS. 3A-3C. The oval shape of the anterior pad 14 increases the contact surface area relative to the circular shape in order to more effectively match the contact surface area of the posterior pad 16. This serves to balance the deformations and contact stresses brought about by each pad 14/16.

With reference to FIGS. 3A-3C, an oval shaped anterior pad 14 is shown. The anterior pad 14 may include a convex inner surface 42 that engages the epicardium when the splint 10 is implanted in the heart H. The anterior pad 14 also includes a circumferential groove 44 to accommodate suture windings to secure a pad covering 46 (shown in phantom). The pad covering 46 may be made of a velour woven polyester material, for example, available under the trade name Dacron™, or other similar suitable material such as expanded polytetrafluoroethylene (ePTFE). The pad covering facilitates ingrowth of the heart wall tissue to secure the pad to the epicardium and thereby prevent long-term, motion-induced irritation thereto. The anterior pad 14 further includes a plurality of inner components (e.g., pins) and channels (not visible) to permit adjustable fixation of the pad 14 to the elongate tension member 12. These features and further aspects of the anterior pad 14 are described in the '049 patent application.

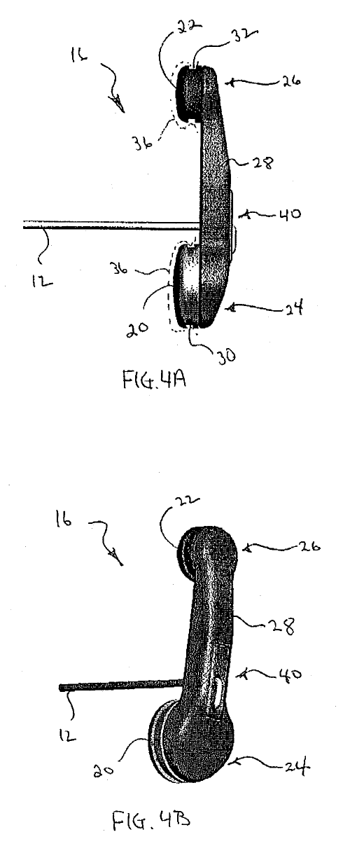

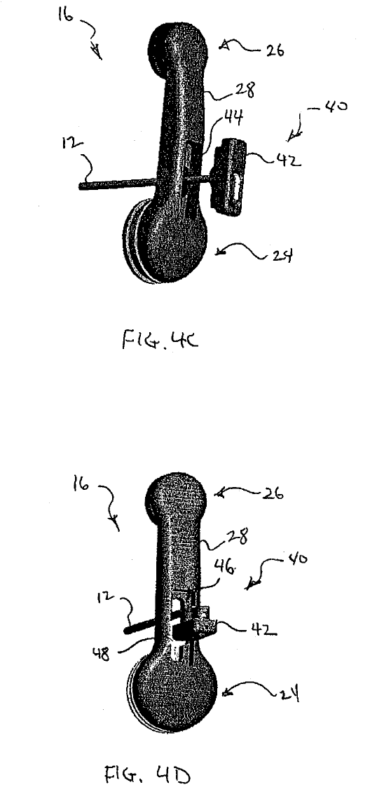

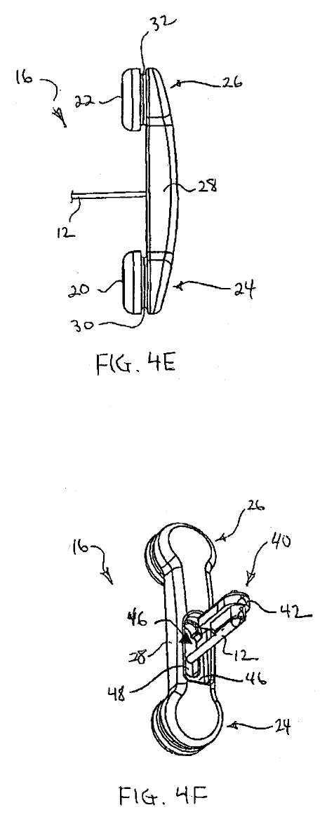

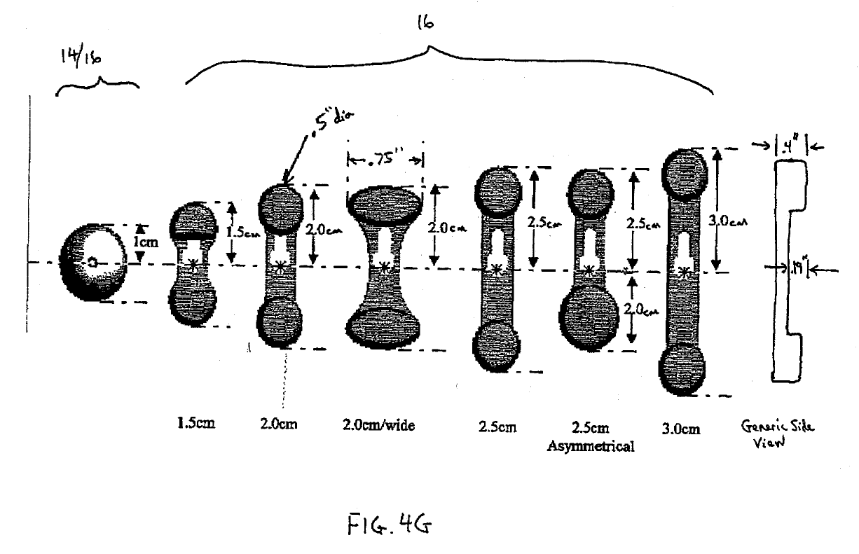

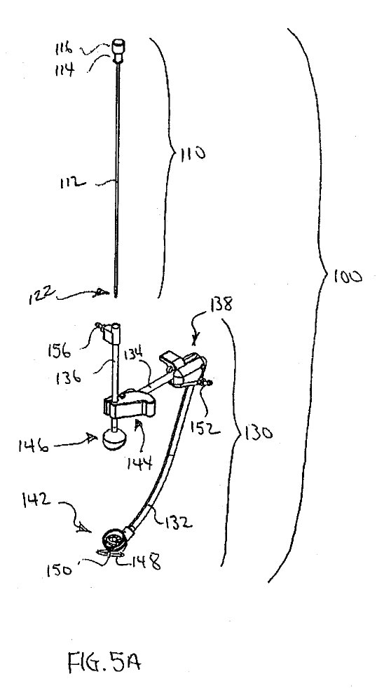

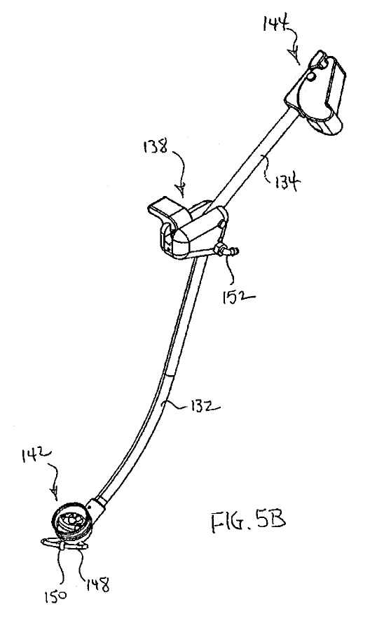

With reference to FIGS. 4A-4F, a posterior pad 16 of the mitral valve splint 10 is shown. In the exemplary embodiments described herein, the posterior pad 16 is a fixed pad, but may be adjustable as well. The posterior pad 16 may define one, two or more contact zones. For example, the posterior pad 16 may define a superior contact zone 22 and an inferior contact zone 20 connected therebetween by bridge 28. The superior contact zone 22 may rest on the epicardial surface of the left ventricle LV, adjacent the annulus AN of the mitral valve MV associated with the posterior leaflet PL. The inferior contact zone 20 may rest on the epicardial surface near the level of the papillary muscles PM of the mitral valve MV, positioned, for example, midway between the papillary muscles PM.

The tension member 10 may intersect the bridge 28 of the posterior pad 16 closer to the inferior end 24 than the superior end 26 as seen in FIG. 4A, for example. The pad 16 thus serves to provide a deformation of a superior portion of the left ventricle LV adjacent the annulus AN of the mitral valve MV, while allowing the tension member 12 to connect to the pad 16 at a position low enough to minimize interference between the tension member 12 and the mitral valve MV structures. To balance the longer moment arm of the bridge 28 exerted by the superior contact zone, the inferior contact zone may have a larger epicardial contact area.

Other posterior pad 16 shapes and sizes are also contemplated, possessing varying numbers and positions of contact zones, possessing varying distances between the contact zones and the tension member, and possessing varying shapes and sizes of contact zones. For example, as shown in FIGS. 4E and 4F, the tension member may alternatively intersect the bridge 28 midway between the superior end 26 and the inferior end 24, and the superior and inferior contact zones 22, 20 may have equal contact surface areas. As a further alternative, the posterior pad 16 may be relatively small, and not necessarily elongated, with the tension member 12 connected to the center of the pad 16 (similar to anterior pad 14), such that the position of the tension member 12 relative to the mitral valve structure is slightly elevated as compared to the embodiment illustrated. Exemplary dimensions and shapes of posterior pad 16 are illustrated in FIG. 4G.

In addition to variations of the design of posterior pad 16, it is also contemplated that variables associated with the position of the pad 16 and forces applied to the pad 16 by the tension member 12 may be selected as a function of, for example, the particular manifestation of mitral valve dysfunction and/or as a function of the particular anatomical features of the patient's heart. These variables may affect the magnitude, area, and/or specific location of displacement of the left ventricular free wall LVFW proximate the mitral valve MV structures (annulus AN, leaflets AL/PL, chordae CT, and/or papillary muscles PM).

With continued reference to FIGS. 4A-4G, the contact zones 20, 22 may have a convex surface that engages the epicardium when the splint 10 is implanted in the heart H. The posterior pad 16 also includes circumferential grooves 30, 32 on each of the contact zones 20, 22 to accommodate suture windings to secure a pad covering 36 (shown in phantom). The pad covering 36 may be made of the same or similar material discussed hereinbefore with reference to anterior pad 14, to facilitate tissue in-growth after implantation.

The posterior pad 16 may incorporate a releasable connection mechanism 40 that allows the pad 16 to be removed from the elongate tension member 12 and replaced, for example, by a different pad with an alternate shape and size, depending on the particular anatomy of the heart H and/or the desired effects on the heart. It may be desirable, for example, to utilize a pad 16 that has a longer bridge 28 with greater spacing between the contact zones 20, 22 to minimize mitral regurgitation. Although the connection mechanism 40 allows the pad 16 to be removed from the tension member 12 and replaced with another pad 16, the position of the pad 16 may remain fixed in that the final position of the pad 16 along the linear aspect of the tension member 12 is fixed, as opposed to the adjustable anterior pad 14 discussed hereinbefore.

The releasable connection mechanism 40 may comprise a block 42 which fits into a recessed region 44 within the pad bridge 28, as best seen in FIGS. 4C and 4F. The block 42 may be fixed to the tension member by one or more pins that penetrate the braided inner cable of the tension member 12, in a manner similar to the connection of the tension member 12 to the anterior pad 14. The recessed region 44 may have a length, width, and height corresponding to the length, width, and height of the block 42, respectively. As best seen in FIGS. 4D and 4F, an inwardly projecting rim 46 is provided at the bottom of the recessed region 44, which prevents the block 42 from moving through the pad bridge 28 in response to tension forces exerted by the tension member 12. An opening 48 is defined by the edge of the rim 46 and is sized such that the block 42 may be passed through the bridge 28 of the pad 16 when the block 42 is lifted away from the bridge 28 and rotated as shown in FIGS. 4D and 4F. A different pad 16, having perhaps a different shape and/or dimensions, may then be connected to the block 42 and tension member 12 by reversing the steps discussed above before final implantation of the splint 10.

It is important to note that while an exemplary embodiment of a mitral valve splint 10 is described above, variations are also considered within the scope of the invention. Mitral valve and cardiac anatomy may be quite variable from patient to patient, and the mitral valve splint design and implant position may vary accordingly. For example, the location of the regurgitant jet may be centered, as shown in FIG. 2A, or may favor one side of the valve opening. Therefore, differences in posterior pad size, pad shape, and overall splint location, for example, may be required to best modify the heart chamber and valve annulus for a particular patient. Steps taken during the delivery of the mitral valve splint 10 are useful to identify and incorporate these design and position variables to suit the particular cardiac anatomy and mitral valve dysfunction.

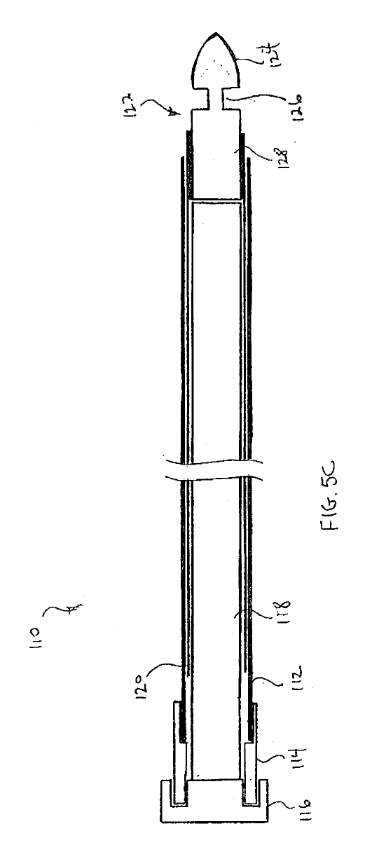

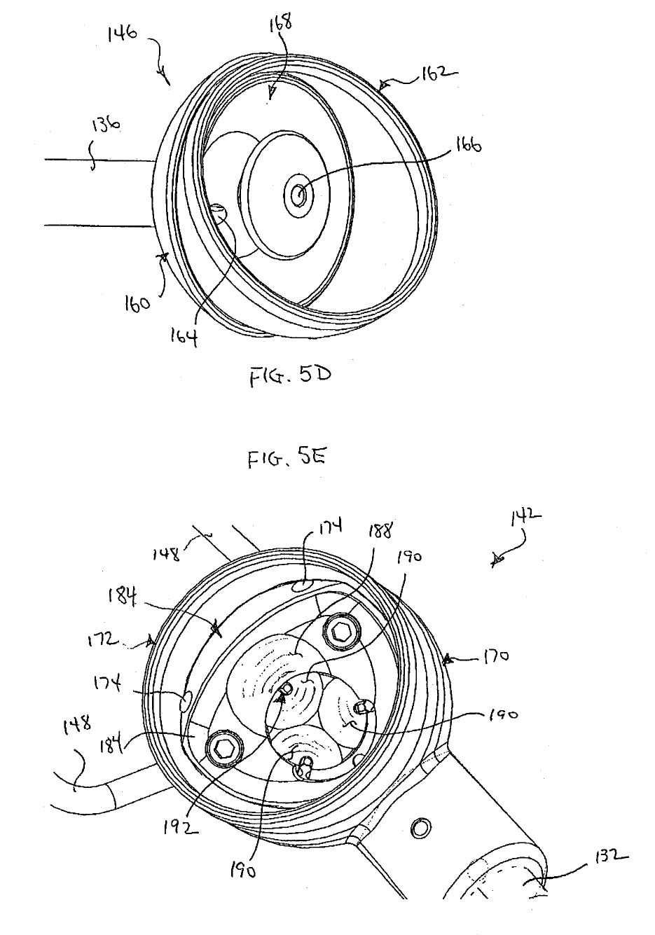

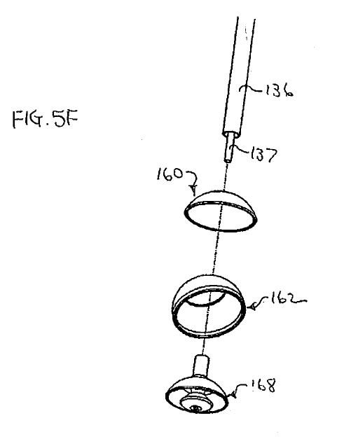

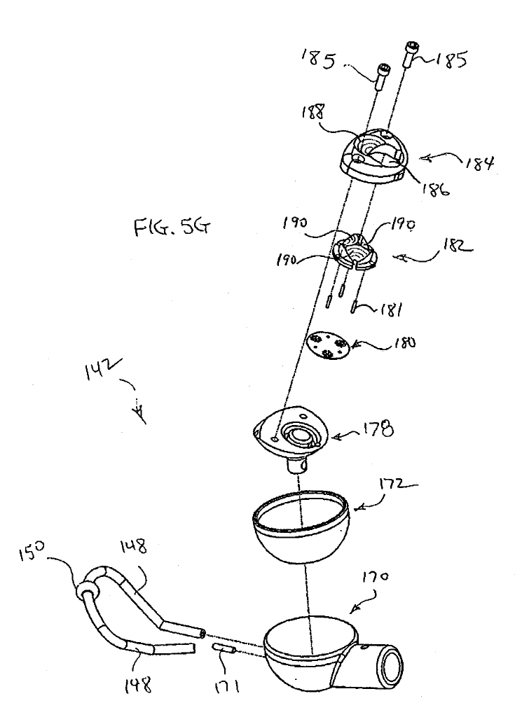

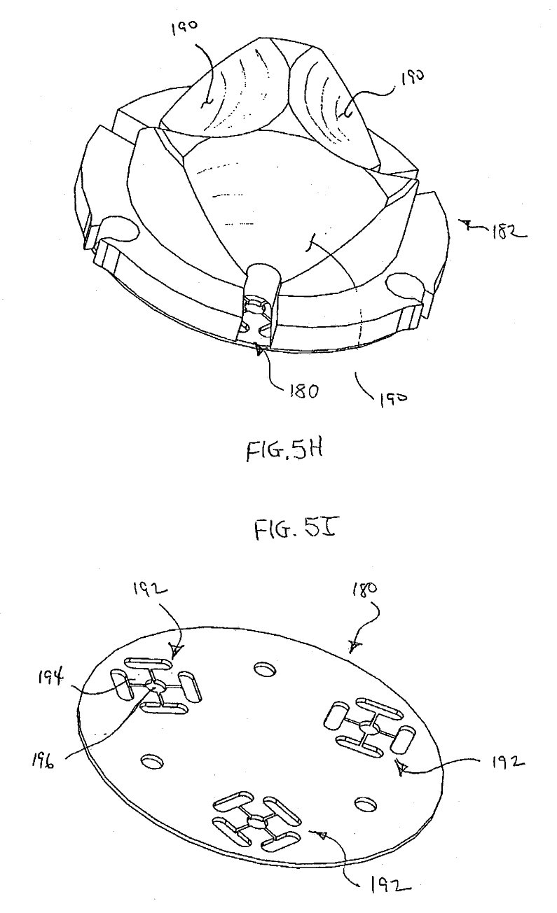

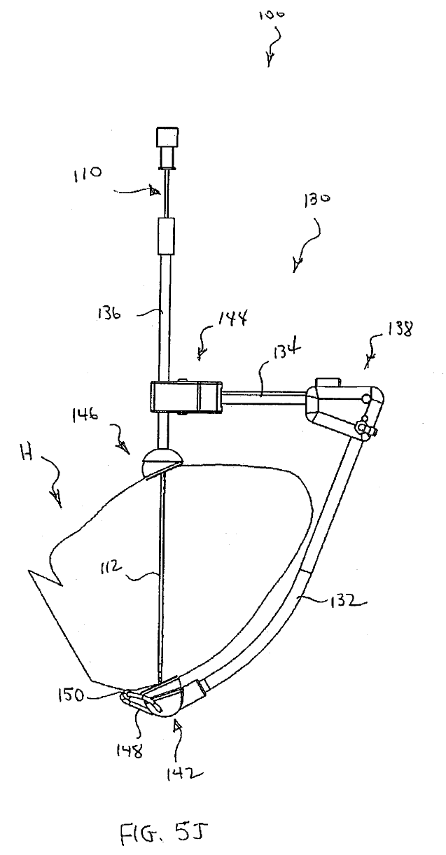

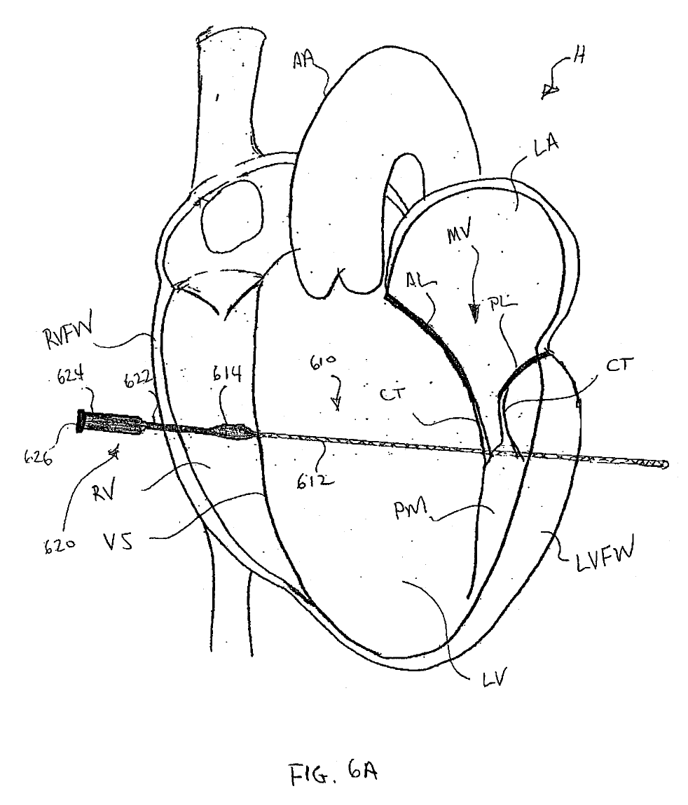

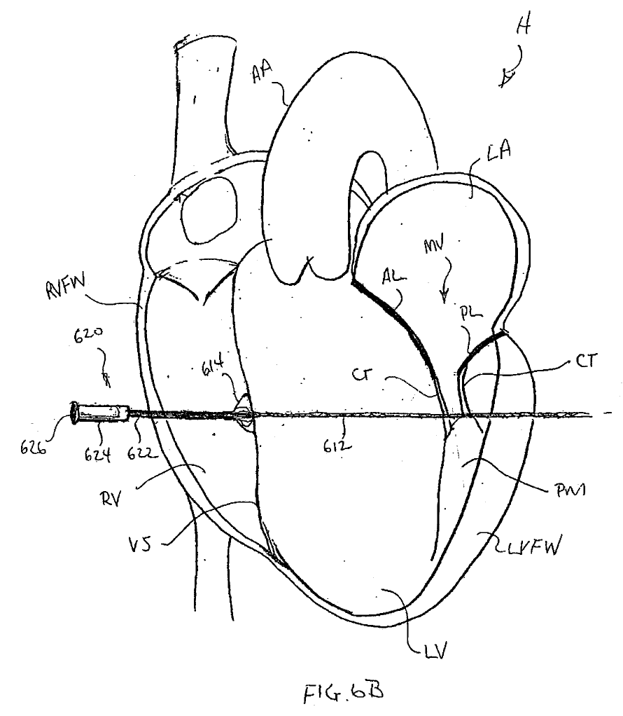

With reference to FIG. 5A, a mitral valve splint delivery system 100 is shown. The mitral valve splint delivery system 100 and associated methods are exemplary, non-limiting embodiments for the delivery of mitral valve splint 10. The mitral valve splint delivery system 100 may include a needle delivery assembly 110, in addition to a positioning and alignment device 130. The positioning and alignment device 130 may be used for identifying and maintaining the desired positions for the subsequent placement of the posterior pad 16 and the anterior pad 14, and the needle delivery assembly 110 may be used for passing the tension member 12 of the splint 10 through the heart H.

The positioning and alignment device 130 may include a posterior arm 132, a swing arm 134, and an anterior arm 136. A lockable hinge 138 allows for relative planar rotation between the posterior arm 132 and the combination of the swing arm 134 and the anterior arm 136. The “closed” position of the hinge 138 is shown in FIG. 5A, and the “open” position of the hinge 138 is illustrated in FIG. 5B. The anterior arm 136 may be joined to the swing arm 134 via a releasable securing clamp 144.

The posterior arm 132 and the anterior arm 136 each may have associated vacuum chambers 142, 146, respectively, for temporary securement of the positioning and alignment device 130 to the epicardial surface of the heart H. At a predetermined spacing from the posterior vacuum chamber 142, an indicator ball 150 may be connected thereto by a fixed dual-arm member 148. The anterior arm 136 may contain a tube defining a lumen for passage of the needle delivery assembly 110 therethrough. The anterior arm 136 and the posterior arm 132 each may have an associated vacuum lumen (not visible) extending therethrough in fluid communication with their respective vacuum chambers 146, 142. Associated fittings 156, 152 may be provided on the anterior arm 136 and the posterior arm 132, respectively, for connecting the corresponding vacuum lumens to a vacuum source (not shown).

With reference to FIG. 5C, the needle delivery assembly 110 may include an outer tube 112, which may be formed of a relatively rigid material such as, for example, a metal (e.g., stainless steel). Other suitable materials also may be used for the outer tube 112. The proximal end of the outer tube 112 may be fixedly connected to a hollow base 114 which may be fixedly or releasably connected to a cap 116. The cap 116 may be fixedly connected to a core member 118 which extends through the outer tube 112 and which may be formed of a relatively rigid material such as, for example, a metal (e.g., stainless steel). A guide tube 120 may be disposed between the outer tube 112 and the inner core member 118. The guide tube 120 may be relatively flexible, kink resistant, and lubricious. For example, the guide tube 120 may be formed of a PTFE liner covered by a metallic braid with a thermoplastic covering such as Nylon. Other suitable materials that permit the guide tube to be relatively flexible, kink resistant, and lubricious also may be used. A tip member 122 including, for example, a sharpened spearhead or bullet-shaped end 124 may be fixedly connected to a distal portion of the guide tube 120 by swaging a short metal tube (not shown) over the guide tube 120 and onto a proximal portion 128 of the tip member 122.

With reference to FIGS. 5D and 5F, the anterior vacuum chamber 146 is shown. The anterior vacuum chamber 146 includes a base housing 160, an articulating rim 162 and a base cover 168. The articulating rim 162 is captured between base housing 160 and base cover 168. A proximal end of the base cover 168 and the base housing 160 are fixedly connected to the anterior arm 136. The articulating rim 162 is movable with respect to the base housing 160, base cover 168 and anterior arm 136, thus allowing the rim 162 to make good contact with the epicardial surface of the heart H and form an effective seal upon application of a vacuum.

In FIG. 5F, the needle tube 137 defining the needle lumen therein is visible extending through the anterior arm tube 136. The lumen of the needle tube 137 opens into the interior of the anterior vacuum chamber 146 at needle port 166. The annular vacuum lumen defined between the needle tube 137 and the anterior arm tube 136 opens into the interior of the anterior vacuum chamber 146 at vacuum port 164.

With reference to FIGS. 5E, 5G, 5H, and 5I, the posterior vacuum chamber 142 is shown. The posterior vacuum chamber 142 includes a base housing 170, an articulating rim 172 and a base cover 178. A proximal end of the base housing 170 is fixedly connected to the posterior arm 132, and the base cover 178 is secured to the base housing 170 by pin 171. The articulating rim 172 is captured between base housing 170 and base cover 178. The articulating rim 172 is movable with respect to the base housing 170, base cover 178 and posterior arm 132, thus allowing the rim 172 to make good contact with the epicardial surface of the heart H and form an effective seal upon application of a vacuum. The base cover 178 includes vacuum ports 174 which are in fluid communication with the interior of the posterior vacuum chamber 142 and which define a fluid path to the vacuum lumen in the posterior arm 132.

The posterior vacuum chamber 142 may include a retainer mechanism. For example, a capture plate 180 may be connected to a rotating insert 182 by connector pins 181. The capture plate 180 and rotating insert 182 are collectively captured between the base cover 178 and a capture plate cover 184, which is secured to the base cover 178 by screws 185. The capture plate 180 and rotating insert 182 are collectively rotatable relative to the base cover 178 and a capture plate cover 184.

The capture plate cover 184 defines an offset opening 186 into which the upper portion of the rotating insert 182 is positioned. The capture plate cover 184 also defines a semi-conical concave slope 188. Similarly, the rotating insert 182 defines a plurality of semi-conical concave slopes 190 that may be individually aligned with the slope 188 on the capture plate cover 184 by indexing (rotating) the rotating insert 182 relative to the capture plate cover 184 such that the semi-conical concave slopes 188, 190 collectively define a conical funnel that serves to guide the needle assembly 110 into the desired dock 192. Thus, if a needle assembly 110 is initially deployed in a first (center) dock 192, and it is desired to re-deploy another needle assembly 110, the rotating insert 182 and capture plate 180 may be collectively rotated relative to the capture plate cover 184 to align a second (auxiliary) dock 192 and its associated semi-conical slope 190 with the semi-conical slope 188 of the capture plate cover 184.

As seen in FIGS. 5H and 51, the capture plate 180 is fixed to the bottom side of the rotating insert 182, with each dock 192 positioned at the bottom of the semi-conical slopes 190. Each dock 192 includes a plurality of deflectable retainer tabs 194 defining a central hole 196. The capture plate 180 may comprise a spring temper stainless steel and the docks 192 may be formed by selectively etching the plate using a photo-etch technique, for example.

As the bullet-shaped tip 124 of the needle assembly 110 is advanced into the posterior vacuum chamber 142, it is guided to a central dock 192 by the funnel collectively defined by slopes 188,190. As the bullet-shaped tip 124 is advanced further into hole 196, the tabs 194 are resiliently deflected away. After the bullet-shaped tip 124 passes the tabs 194 and the distal end thereof is stopped by base cover 178, the tabs 194 resiliently spring back into the detent space 126 of the tip assembly 122, serving to lock the position of the tip assembly 122 and guide tube 120 relative to the posterior vacuum chamber 142.

Those skilled in the art will recognize that the positioning and alignment device 130 may be formed of a variety of materials and may have a variety of dimensions depending on, for example, the conditions of use and anatomical variability. By way of example, not limitation, the posterior arm 132, swing arm 134 and anterior arm 136 may be formed of stainless steel tubing. The connective elements (pins, screws, etc.) may also be formed of stainless steel. The rims 162, 172 of the anterior and posterior vacuum chambers 146, 142, respectively, may be formed of clear polycarbonate, or other similar suitable material, to facilitate visualization of the epicardial surface thereunder. The dual-arm 148 and the indicator ball 150 may be formed of PEEK with a stainless steel core wire running therethrough. The remaining components of the positioning and alignment device 130 may be formed of a polymeric material such as acetyl available under the trade name Delrin™. The vacuum lines connecting the fittings 152/156 to a vacuum source may comprises polyether block amide tubes with stainless steel coil windings therein. Other suitable materials may be used and are contemplated as being within the scope of the invention.

Also by way of example, not limitation, the posterior arm 132 may have a length of approximately 18 cm, the swing arm 134 may have a length of approximately 10 cm, and the anterior arm may have a length to accommodate approximately 5 cm to 13 cm of adjustable distance between the anterior vacuum chamber 146 and the posterior vacuum chamber 142. These exemplary dimensions have been found to accommodate a wide variety of anatomical sizes and variations. The needle assembly 110 may have a length of approximately 46 cm to traverse the heart H and provide sufficient length and flexibility for manipulation around the heart. The anterior vacuum chamber 146 and the posterior vacuum chamber 142 may have outside diameters of approximately 2 cm to provide adequate yet atraumatic holding power on the epicardium. Other suitable dimensions may be selected depending on a patient's particular anatomy, for example.

In use, the positioning and alignment device 130 is initially in the open position. The posterior arm 132 may be positioned through a thoracotomy (e.g. a median sternotomy), along the posterior aspect of the heart H and generally aligned with the long axis of the left ventricle LV. The indicator ball 150 may be positioned in the AV groove, by visual or tactile cues, or a combination of such cues. During this procedure, the heart H may be manipulated to facilitate direct visualization. The predetermined distance between the indicator ball 150 and the posterior vacuum chamber 142 places the vacuum chamber 142 in a desired position relative to the annulus AN of the mitral valve MV. The posterior vacuum chamber 142 is activated by applying a vacuum thereto, securing the chamber 142 to the epicardial wall is the desired position. The center of the posterior vacuum chamber 142 now corresponds to the future location of the intersection of the tension member 12 with the left ventricular LV chamber wall.

Assessment of the position of the posterior vacuum chamber 142 relative to internal mitral valve MV structures such as leaflets AL, PL, papillary muscles PM, and regurgitant jet may be performed with ultrasonic imaging such as trans-esophageal or epicardial echocardiography. The position of the posterior vacuum chamber 142 may be visualized on the echocardiogram by observing the portion of the left ventricular free wall LVFW that is less dynamic than the remaining portions thereof, rendered so by the dampening effect of the posterior vacuum chamber 142 fixed thereto. Mechanical manipulation of the positioning and alignment device 130 may also be performed to assess the functional impact of this position on the mitral valve regurgitation, as the heart is still beating. For example, the positioning and alignment device 130 may be pivoted about the posterior vacuum chamber 142 to drive the indicator ball 150 into the AV groove, thereby exerting an inward force on the annulus AN of the mitral valve MV. If the position is not optimal, the vacuum may be de-activated, and the posterior vacuum chamber 142 may be repositioned as desired. Conveniently, the posterior vacuum chamber 142 will leave a pucker mark on the epicardium at the initial position thereof, which may serve as a reference mark for repositioning.

The anterior arm 136, initially disconnected from the swing arm 134, is then manipulated to position the anterior vacuum chamber 146 on the epicardial surface of the heart, corresponding to the subsequent desired position of the anterior anchor pad 14. As the anterior arm is manipulated, echocardiographic information pertaining to the right ventricle RV and nearby tricuspid valve TV may be assessed and utilized to help find a desired position for the anterior vacuum chamber 146. Once in a desired position, the anterior vacuum chamber 146 is activated by application of vacuum, temporarily securing anterior vacuum chamber 146 to the epicardial surface of the heart. The swing arm 134 is then rotated into position to allow for the securing clamp 144 to clamp onto the anterior arm 136. The anterior arm 136 preferably is long enough (e.g., 5 to 15 cm) to allow for significant variations in heart diameters from patient to patient.

Both vacuum chambers 142, 146 are now securely positioned on the epicardial surface of the heart, in positions which will correspond to the anterior and posterior anchor pads 14, 16. The needle delivery assembly 110 now may be inserted through the passage lumen provided in the anterior arm 136, through the anterior vacuum chamber 146, across the heart and into the posterior vacuum chamber 142. The positioning and alignment device 130, with the needle delivery assembly 110 fully inserted through the heart chamber, is illustrated in FIG. 5J.

As the needle delivery assembly 110 is passed into the posterior vacuum chamber 142, the circumferential detent 126 on the tip assembly 122 engages with the retention mechanism of the posterior vacuum chamber 142. Once the needle delivery assembly 110 is locked in position in the central dock 192, the cap 116 and base 114 are pulled proximally from the anterior arm 136, thus removing the outer tube 112 and core member 118 from the needle delivery assembly 110. The tip assembly 122 and guide tube 120 are thus left in position across the heart chamber and define the path that will be taken by the tension member 12 through the heart H.

The vacuum to the anterior and posterior chambers 146, 142 may then be interrupted, allowing the positioning and aligning device 130 to be removed from the surface of the heart. As the positioning and aligning device 130 is removed from the heart, the tip assembly 122 and guide tube 120 remain engaged with the posterior vacuum chamber 142, bringing the tip assembly 122 and distal end of the guide tube 120 to an easily accessible location nearer the anterior side of the heart H. The tip assembly 122 may then be removed from the guide tube 120, such as by using a scissors, for example. The positioning and aligning device 130 is then removed from the surgical field, leaving only the guide tube 120 positioned across the heart chamber in the desired position for delivery of the mitral valve splint 10.

If necessary or desired, it is possible to reposition the guide tube 120. The positioning and aligning device 130 at this stage has the tip 122 from the prior needle delivery assembly 110 in the central dock 192. This tip 122 may be rotated out of position, bringing one of the auxiliary docks 192 into alignment with the slope 188 of the capture plate cover 184 as described hereinbefore. The positioning and aligning device 130 may then be repositioned on the heart H as described before, and a different needle delivery assembly 110 may then be delivered in a new position following the same steps described above.

Once the guide tube 120 is deemed in an appropriate position, the mitral valve splint 10 may be delivered in a manner similar to the method described in the copending U.S. application Ser. No. 09/680,435, filed Oct. 6, 2000, entitled METHODS AND DEVICES FOR IMPROVING MITRAL VALVE FUNCTION (hereinafter the '435 application), the entire disclosure of which is incorporated by reference. The tension member 12 is provided with the posterior (fixed) pad 16, or at least the block 42 of the releasable connection mechanism 40, connected thereto. The tension member 12 may include a leader section (not shown) that is advanced into the now accessible posterior (distal) end of the guide tube 120. Once the leader of the tension member 12 emerges from the anterior (proximal) end of the guide tube 120, the leader of the tension member 12 and the guide tube 120 are pulled proximally, placing the posterior anchor pad 16 in position on the epicardium. The anterior (adjustable) pad 14 is then positioned on the tension member 12. A measuring and tightening device such as that described in U.S. Pat. No. 6,260,552 to Mortier et al., the disclosure of which is incorporated herein by reference, may be used to adjust the spacing of the anterior and posterior pads 14, 16 to an optimum distance. Mitral valve function may be observed with appropriate diagnostic techniques such as transesophageal echocardiography (TEE) to assist in determining the appropriate distance between the anterior and posterior pads 14, 16 and the appropriate tightness of the splint 10.

Once the splint 10 is appropriately tightened, the anterior pad 14 is secured to the tension member 12, similar to the method described in the '435 application, incorporated herein. At any time during delivery of the splint 10, the posterior pad 16 may be switched to a pad of a different shape or size, as described hereinbefore, by utilizing the releasable connection mechanism 40. Once the proper posterior pad 16 is in place and the desired mitral valve function is established and confirmed using an appropriate diagnostic method, the thoracotomy may be closed.

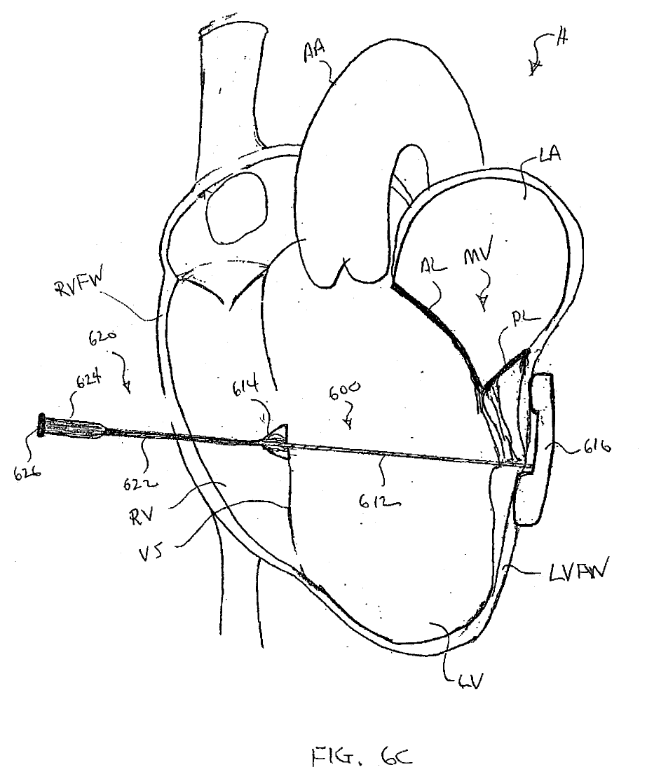

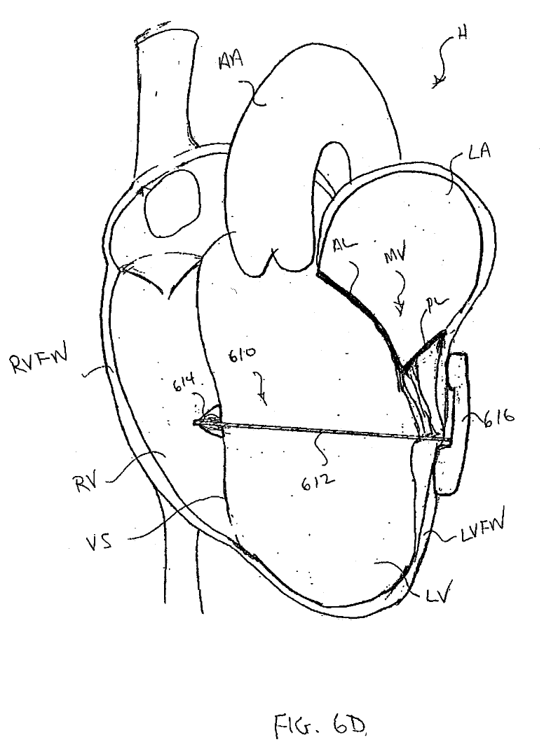

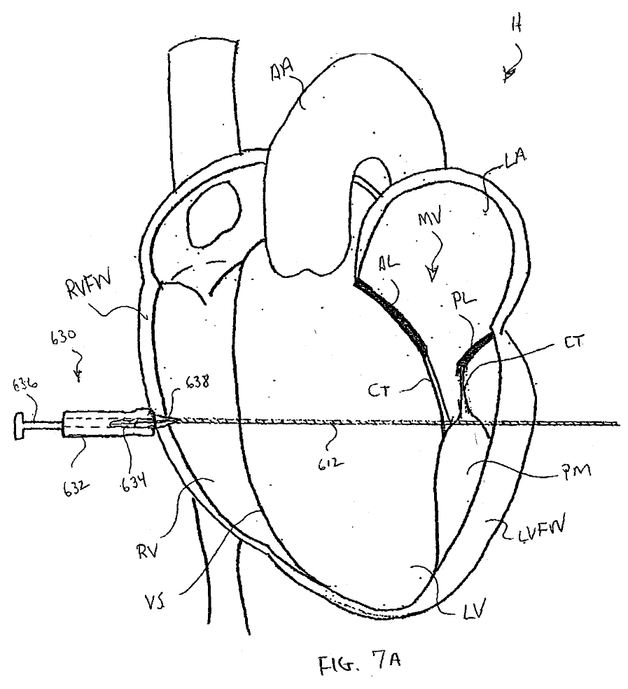

With reference to FIGS. 6A-6D, exemplary embodiments of a septal mitral valve splint 610, septal delivery system and septal delivery method are schematically illustrated, which may be similar to that described with reference to the epicardial mitral valve splint 10, except as apparent from the drawings and related discussion. As best seen in FIG. 6D, the septal mitral valve splint 610 generally includes a tension member 612, a septal anchor 614, and a posterior (epicardial) pad 616. Tension member 612 may be similar to tension member 12, and posterior pad 616 may be similar to posterior pad 16.

A general difference between the septal approach illustrated in FIGS. 6A-6D and the epicardial approach illustrated in FIGS. 1A-1C is that the anterior (epicardial) pad 14 has been replaced by a septal anchor 614 that may be located more superiorly, thus altering the force vector of the tension member 12. The septal approach may be better suited for certain types of mitral valve dysfunction than the epicardial approach. However, as with the epicardial approach, the septal approach causes local deformation of the annulus AN of the mitral valve MV and brings the posterior leaflet PL in better apposition to the anterior leaflet AL. In addition, one or both papillary muscles PM may be repositioned, further facilitating leaflet apposition and minimizing mitral valve regurgitation.

To facilitate delivery of the septal splint 610, a balloon-tipped probe 620 may be utilized. The probe 620 may include an elongate shaft 622 having a length sufficient to extend across the right ventricle RV to the ventricular septum VS as shown in FIG. 6A. A handle 624 having an inflation port 626 is connected to the proximal end of the shaft 622 and a balloon 614 is detachably connected to the distal end of the shaft 622. The shaft 622 may include an inflation lumen that defines a fluid path between the inflation port 626 and the interior of the balloon 614 to permit the balloon 614 to be selectively inflated and deflated by utilizing a syringe (not shown) or other suitable inflation device connected to the port 626. The balloon 614 may be formed of PET or other similar suitable material, and may be fixedly connected to the proximal end of the tension member 612. However, the shaft 622 may optionally include a tension member lumen to accommodate the tension member 612 therein. The tension member lumen may extend through the balloon 614 and all or a portion of the elongate shaft 622 and handle 624.

In use, a guide tube (not shown in FIGS. 6A-6D), similar to guide tube 120 discussed above, may be delivered across the right ventricle RV and left ventricle LV utilizing the delivery system 100 and related method described previously, but with a different orientation as shown in FIG. 6A. The tension member 612, with its proximal end fixedly connected to the balloon 614, may then be threaded through the guide tube from the anterior side to the posterior side, and the guide tube may be subsequently removed. The distal (posterior) end of the tension member 612 may be pulled posteriorly, to pull the probe 620 through the right ventricular free wall RVFW and right ventricle RV until the balloon 614 abuts the ventricular septum VS as shown in FIG. 6A.

A syringe (not shown) or other suitable inflation device may then be connected to the port 626 of the handle 624. The syringe may contain a curable inflation fluid such as, for example, a bone cement. The syringe may then be used to inflate the balloon 614 with the curable material as seen in FIG. 6B. The inflated balloon 614 may have a conical geometry, for example, that provides a larger surface area against the ventricular septum VS. The tension member 612 may be embedded in the curable material residing in the balloon 614 to provide a more effective bond therebetween. A posterior pad 616 may then be connected to the distal end of the tension member 612. After the material in the balloon 614 has cured, the posterior pad 616 may be adjusted on the tension member 612 to adequately tighten the splint 600 and force the leaflets AL, PL into full apposition, as shown in FIG. 6C. The balloon 614 may then be detached from the shaft 622 and the probe may be removed as shown in FIG. 6D.

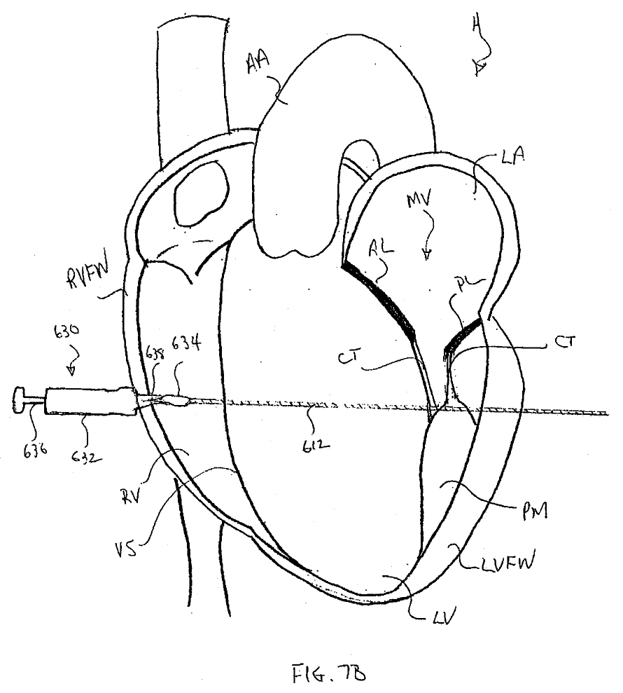

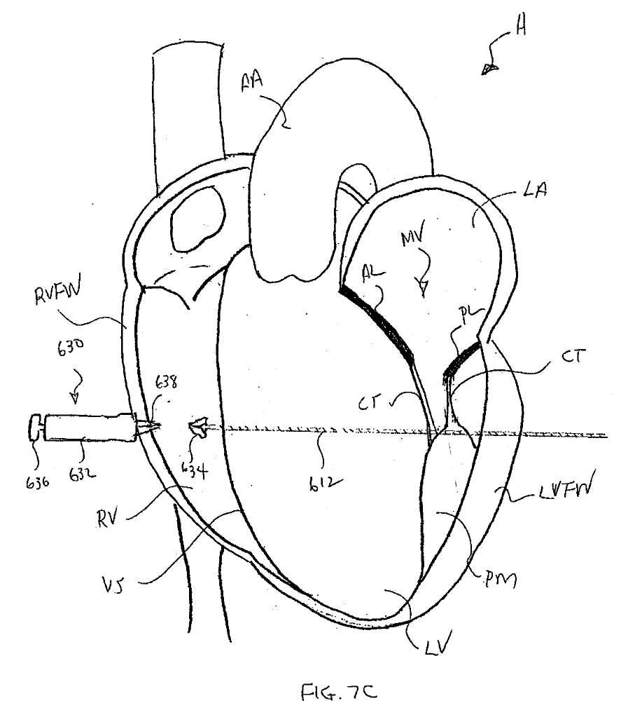

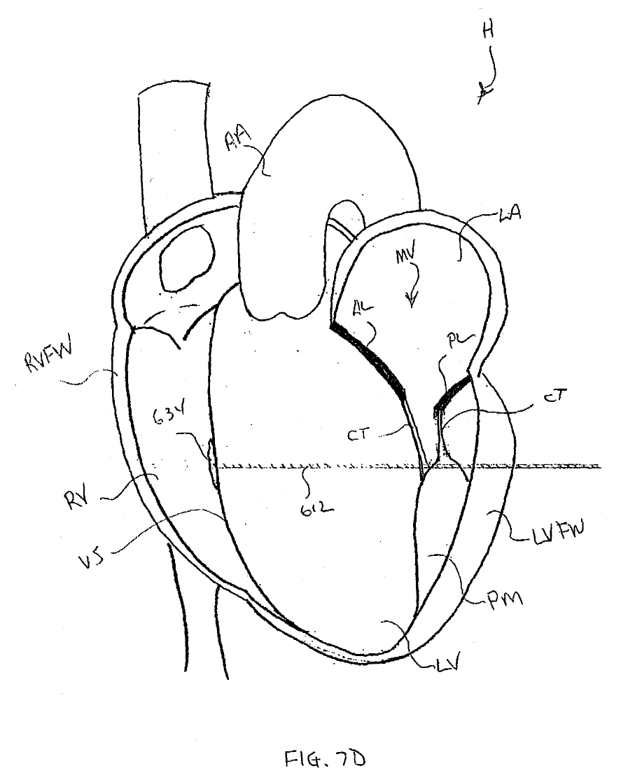

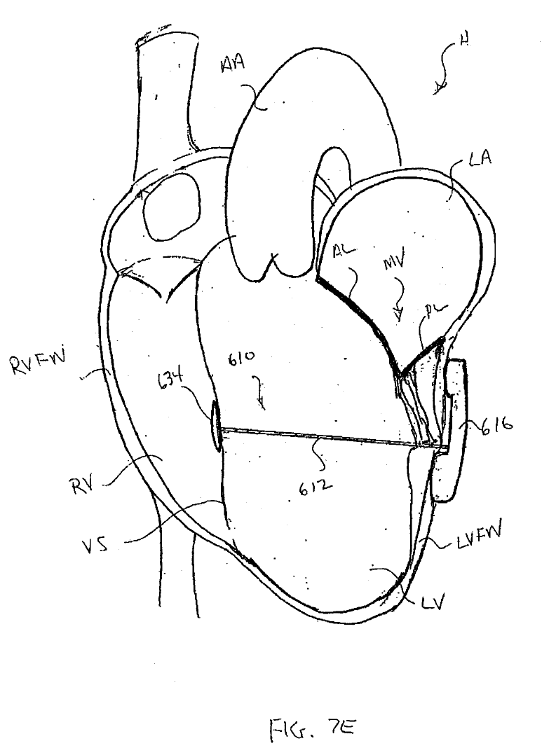

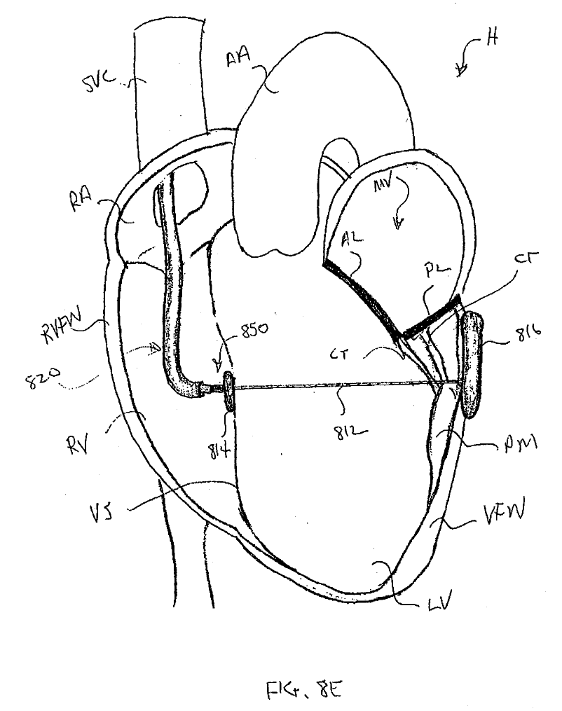

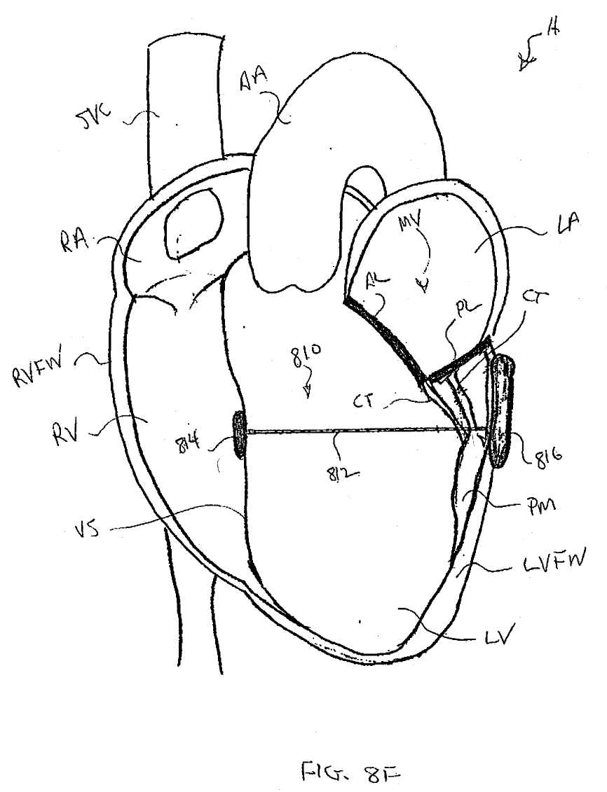

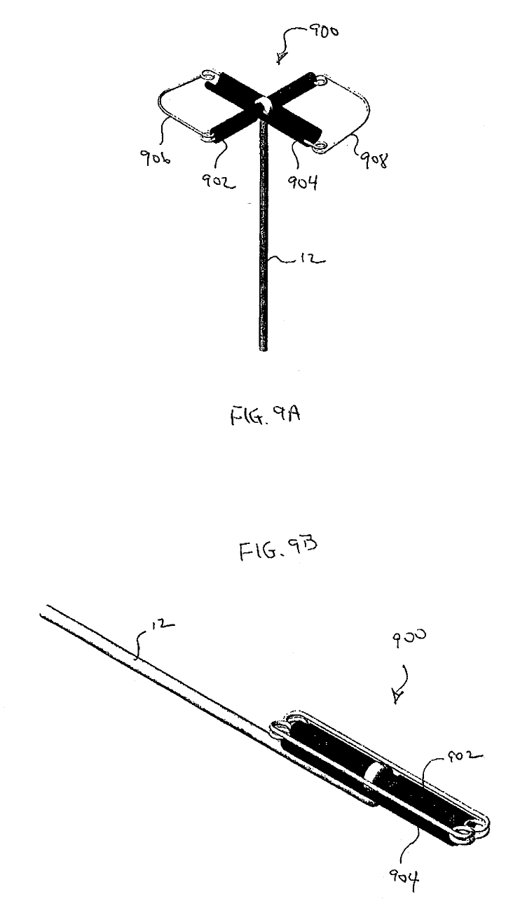

With reference to FIGS. 7A-7E, schematic illustrations of exemplary embodiments of an alternative septal pad 634 and delivery system are shown for the mitral valve splint 610 described with reference to FIGS. 6A-6D. The primary difference between the septal approach illustrated in FIGS. 7A-7E and the septal approach illustrated in FIGS. 6A-6D is that the septal balloon pad 614 has been replaced by a self expanding septal pad 634. Other aspects may remain the same or similar. As best seen in FIG. 7E, the septal mitral valve splint 610 generally includes a tension member 612, a septal anchor 634, and a posterior (epicardial) pad 616. The self expanding septal pad 634 may comprise any of the devices described with reference to FIGS. 9A-9D, for example, and may be fixedly connected to the proximal (anterior) end of the tension member 612.

To facilitate delivery of the self expanding septal pad 634, a delivery probe 630 may be utilized. Delivery probe 630 may include a barrel 632 defining a chamber therein which contains the self expanding septal pad 634 in a collapsed mode. A plunger 636 may extend into a proximal portion of the barrel 632. An expandable and sharpened tip 638 capable of penetrating the heart wall may be provided at the distal end of the barrel 632. Actuation of the plunger 636 in the distal direction with respect to the barrel 632 causes the self expanding septal pad 634 to be pushed into and through the tip 638, which may expand to accommodate the self expanding septal pad 634 therein.

In use, a guide tube similar to guide tube 120 (not shown) may be delivered across the right ventricle RV and left ventricle LV utilizing the delivery system 100 and related method described previously, but with a different orientation as compared to the orientation shown in FIG. 1A. The tension member 612, with its proximal end fixedly connected to the self expanding septal pad 634, may then be threaded through the guide tube from the anterior side to the posterior side, and the guide tube may be subsequently removed. The distal (posterior) end of the tension member 612 may be pulled posteriorly to pull the tip 638 of the probe 630 so that the tip 638 penetrates the right ventricular free wall RVFW as shown in FIG. 7A. The tension member 612 may continue to be pulled posteriorly until the self expanding septal pad 634 exits the tip 638 of the probe 630, as shown in FIG. 7B, enlarges to its expanded mode as shown in FIG. 7C, and abuts the ventricular septum VS as shown in FIG. 7D. A posterior (adjustable) pad 616 may then be connected to the distal end of the tension member 612 and adjusted to adequately tighten the splint and force the leaflets AL, PL into full apposition, as shown in FIG. 7E.

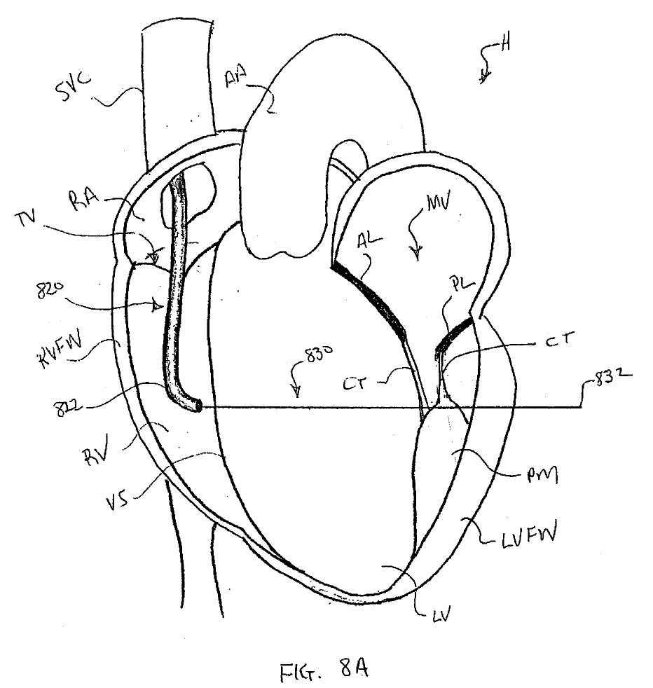

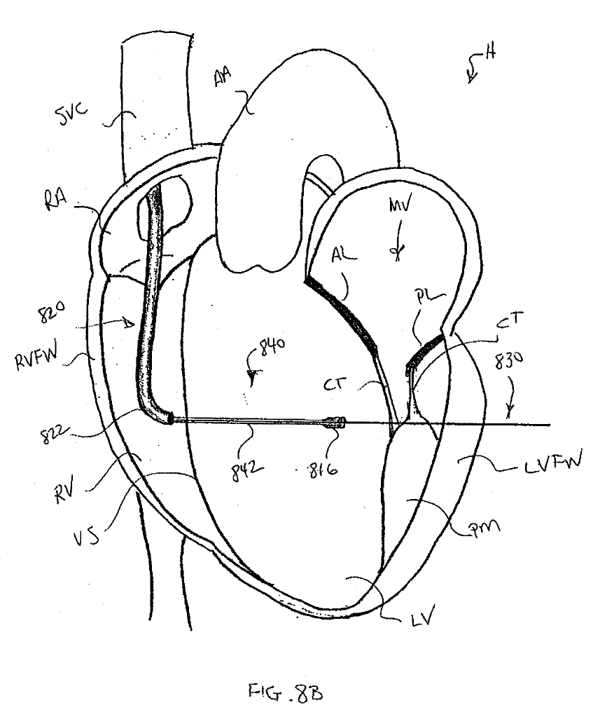

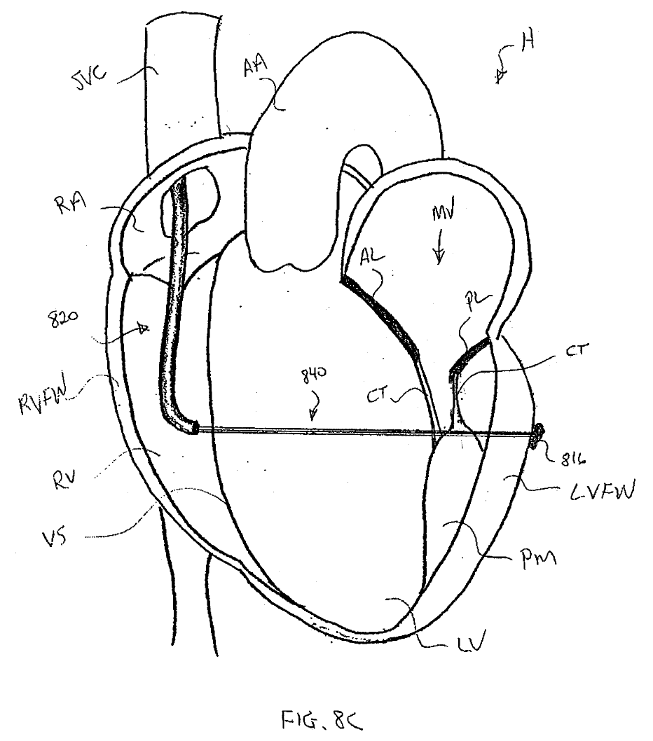

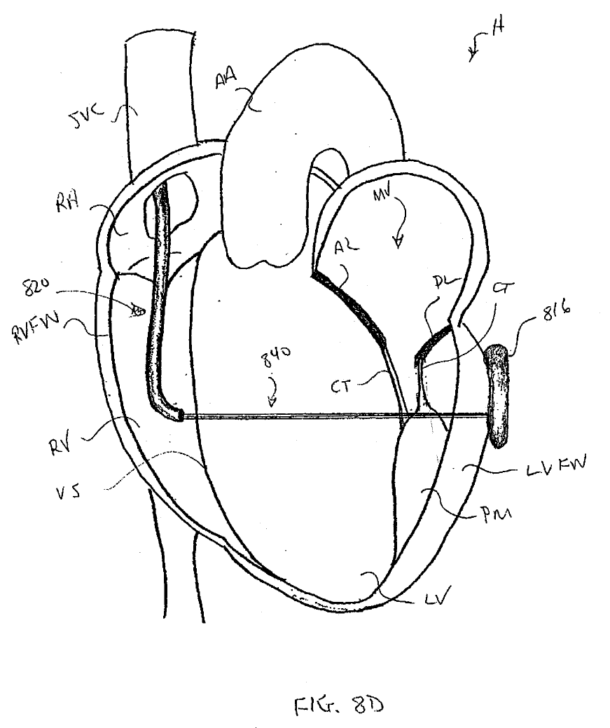

With reference to FIGS. 8A-8F, schematic illustrations of exemplary embodiments of yet another septal splint 640 and delivery method are shown. The septal approach illustrated in FIGS. 8A-8F is generally different than those described hereinbefore in that it is an endovascular approach, but other aspects may remain the same or similar to those described with reference to FIGS. 6A-6D. More details of an endovascular approach may be found in U.S. patent application Ser. No. 09/679,550, entitled ENDOVASCULAR SPLINTING DEVICES AND METHODS, the entire disclosure of which is incorporated herein by reference.

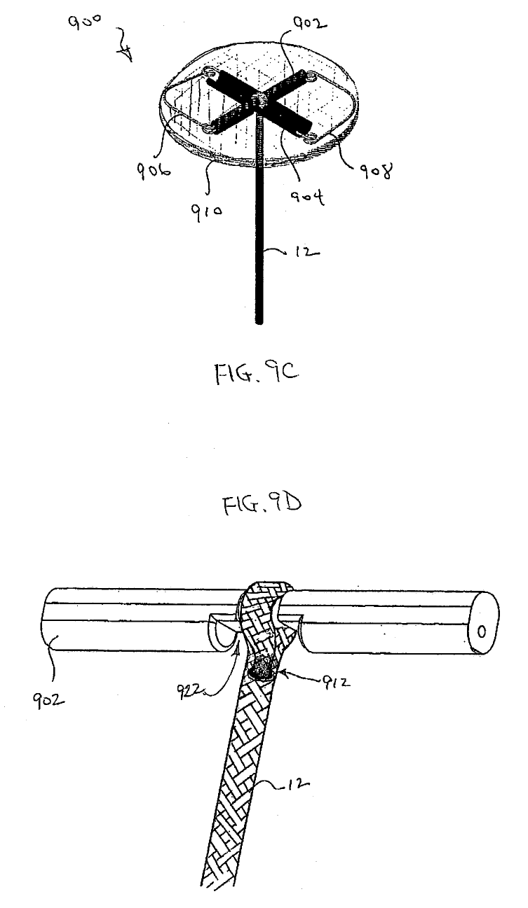

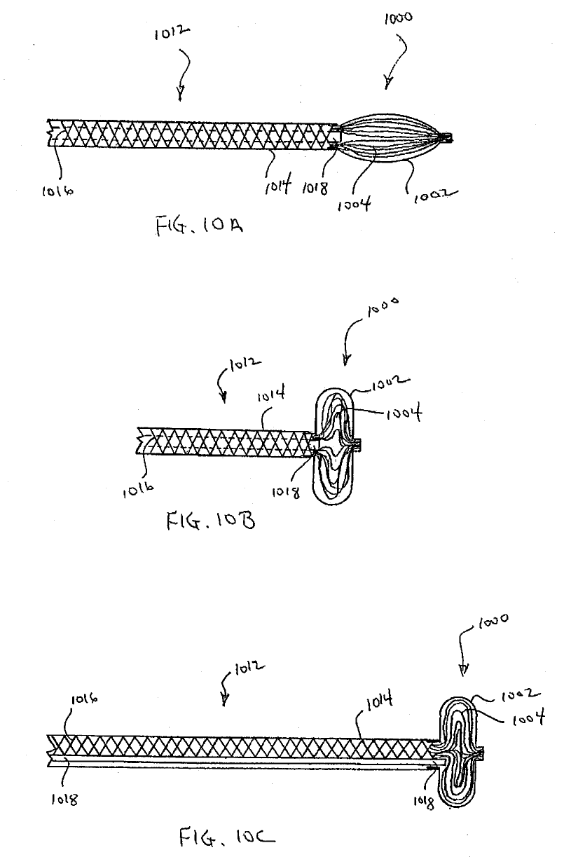

As best seen in FIG. 8F, the endovascular septal mitral valve splint 810 generally includes a tension member 812, a septal pad 814, and a posterior (epicardial) pad 816. The septal and epicardial pads 814, 816 may comprise, for example, any of the devices described with reference to FIGS. 10A-10C. Tension member 812 may be the same as or similar to tension member 12.

In use, a guide catheter 820 may be navigated through a patient's vascular system until the distal end thereof resides within the right ventricle RV. For example, the guide catheter 820 may be navigated from the peripheral veins in the arm to the superior vena cava SVC, through the right atrium RA, past the tricuspid valve TV, and into the right ventricle RV. The distal end of the guide catheter 820 includes a curved portion 822 to direct the distal end of the guide catheter 820 at the ventricular septum VS. Once the guide catheter 820 is in this position, a guide wire 830 may be inserted through the guide catheter 820. A tissue penetrating tip (e.g., sharpened tip) 832 of the guide wire 830 may pass through the ventricular septum VS, across the left ventricle LV, and through the left ventricular free wall LVFW as shown in FIG. 8A.

A balloon-tipped catheter 840 may then be passed over the guide wire 830 as shown in FIG. 8B. The balloon catheter 840 includes an elongate shaft 842 that extends through the guide catheter 820. A detachable balloon 816 may be connected to the distal end of the shaft 842, and may be formed of PET, for example. The elongate shaft 842 may include a guide wire lumen and an inflation lumen (not visible). The inflation lumen is in fluid communication with the balloon 816 and an inflation port (not visible) connected to a proximal end of the shaft 842. The guide wire lumen may extend through the balloon 816 and all or a portion of the shaft 842. The tension member 812 (not visible) is fixedly connected to the balloon 816 and extends proximally in the shaft 842 of the catheter.

The balloon catheter 840 may then be urged distally over the guide wire 830 until the balloon traverses the left ventricular free wall LVFW as shown in FIG. 8C, and the guide wire 830 may be removed. A syringe (not shown) or other inflation device may then be connected to the inflation port at the proximal end of the catheter 840. The syringe may contain a curable inflation fluid such as, for example, a bone cement. The syringe may then be used to inflate the balloon 816 with the curable material as seen in FIG. 8D. The balloon 816 may have an asymmetric inflated geometry, for example, that extends superiorly adjacent the annulus AN of the mitral valve MV, and that provides a large atraumatic surface area against the epicardial surface as seen in FIG. 8D. Alternatively, the balloon 816 may have a symmetric inflated geometry. Once cured, the catheter shaft 842 my be detached from the balloon 816, leaving the balloon 816 as the posterior epicardial pad and leaving the tension member 812 extending across the left ventricle as shown in FIG. 8E.

Using the tension member 812 as a substitute for the guide wire 830, another balloon-tipped catheter 850 may then be passed over the tension member 812. The balloon catheter 850 is similar to balloon catheter 840, except that balloon 814 may be secured to the tension member 812 upon curing. The second balloon catheter 850 may be urged distally until the balloon 814 engages the ventricular septum VS is inflated with a curable material. With the posterior balloon 816 in the desired location and the distal end of the tension member 812 fixed thereto, the tension member 812 may be pulled proximally while pushing on the second balloon catheter 850 to force the leaflets AL, PL into full apposition as shown in FIG. 8E. The balloon 814 of the second balloon catheter 850 is allowed to cure, thus securing the tension member 812 to the balloon 814, which then becomes the septal pad 814. The balloon 814 is detached from the remainder of the catheter 850. The tension member 812 may then be cut adjacent the proximal side of the septal pad 814, and the catheters 820, 850 may be removed, thus leaving splint 810 implanted as shown in FIG. 8F.

With reference to FIGS. 9A-9D, perspective views of a self expanding pad 900 and associated components are shown. The self expanding pad 900 may be used with the septal mitral valve splints of FIGS. 6-8, for example, as discussed above. The self expanding pad 900 is expandable between a collapsed delivery configuration as shown in FIG. 9B, and an expanded deployed configuration as shown in FIG. 9A. The small profile (diameter) of the self expanding pad 900 in the collapsed configuration permits the pad 900 to be delivered through a low-profile catheter or probe as described with reference to FIGS. 6-8, while the large profile of the self expanding pad 900 enables the pad to effectively and atraumatically engage the epicardium or septum, while resisting being pulled therethrough by the tension member 12.

Self expanding pad 900 includes a first arm 902 and a second arm 904 that pivot at their midpoints. The tension member 12 is fixedly connected to the first arm 902 and extends through a central hole in the second arm 904, thus pivotally connecting the two arms 902, 904. Two spring members 906, 908 are connected to the ends of the first and second arms 902, 904 as shown, to provide a biasing force on the arms 902, 904 rendering them self-expandable. The two spring members 906, 908 may be formed of spring tempered stainless steel, for example, or other suitable material. The first arm 902 and the second arm 904 may be formed of a stainless steel hypotube stock, for example, or other suitable material.

The first arm 902 may have a circular cross-section and the second arm 904 may be crimped to define a c-shaped or u-shaped cross-section. With this geometry, the first arm 902 rests in the second arm 904 (in the collapsed configuration) to create a toggle between the collapsed configuration and the expanded configuration. The first arm 902 defines a central recess 922 (visible in FIG. 9D) that is slightly wider than the width of the second arm 904 to accommodate and lock the second arm 904 in the expanded configuration.

As shown in FIG. 9C, the self expanding pad 900 may include a covering 910 formed of a velour woven polyester material, for example, available under the trade name Dacron™, or other similar suitable material such as, for example, expanded polytetrafluoroethylene (ePTFE). The covering 910 facilitates ingrowth of the heart wall tissue to secure the pad 900 to the epicardium or septum and thereby prevent long-term, motion-induced irritation thereto.

As shown in FIG. 9D, the tension member 12 may be connected to the first arm 902 by a tubular braid connection 912. In this exemplary embodiment, the inner cable of the tension member 12 may comprise a tubular braid, with one end of the tubular braid wrapped around the recess 922 of the first arm 902 and inserted into a hole at connection 912. When tensile forces are applied to the connection 912, the tubular braid constricts thereby locking down on the end inserted through the hole, similar to a Chinese finger lock.

With reference to FIGS. 10A-10C, perspective views of an expandable balloon pad 1000 and associated components are shown for use with the septal mitral valve splints of FIGS. 6-8, for example. The expandable balloon pad 1000 is connected to the distal end of a catheter shaft 1012, that may be detachable or that may serve as tension member 12. The expandable balloon pad 1000 includes an outer balloon 1002 formed of a thin polymer such as PET, for example. The distal end of the outer balloon 1002 is closed and sealed about the distal end of cable filaments 1004. The cable filaments 1004 may comprise the same or similar filaments forming the cable core of the tension member 12, for example. The filaments 1004 may extend proximally from the sealed distal end of the balloon 1002 and into the catheter shaft 1012.

The catheter shaft 1012 includes an outer tube 1014 to which the proximal end of the balloon 1002 is bonded and sealed. The catheter shaft 1012 also includes an inner tube 1018 disposed in the outer tube 1014 which defines an inflation lumen extending therethrough in fluid communication with the interior of the balloon 1002. The shaft 1012 may include a braid reinforcement 1016 carried in or under the outer tube 1014 to provide the same properties as the tension member 12. The braid reinforcement 1016 may comprise a continuation of the filaments 1004 extending from the balloon 1002. Alternatively, braid reinforcement 1016 may comprise a separate component and the proximal end of the filaments 1004 may be connected to the bond site between the balloon 1002 and outer tube 1014. If the braid reinforcement 1016 comprises a continuation of the filaments 1004 extending from the balloon 1002, the filaments 1004 forming the braid may extend coaxially around the inner tube 1018 as shown in FIGS. 10A and 10B, or extend adjacent the inner tube 1018 as shown in FIG. 10C.

A syringe (not shown) or other inflation device may be connected to the proximal end (not shown) of the shaft 1012 to communicate with the inflation lumen of the inner tube 1018. The syringe may contain a curable inflation fluid such as a bone cement. The syringe may then be used to inflate the balloon 1002 with the curable material as seen in FIGS. 10B and 10C. The inflated balloon 1002 may have a disc geometry, for example, that provides a larger surface area against the epicardium or septum. The filaments 1004 may be embedded in the curable material residing in the balloon 1002 to provide a more effective bond therebetween.

Those skilled in the art will recognize that the present invention may be manifested in a variety of forms other than the specific embodiments described and contemplated herein. Accordingly, departures in form and detail may be made and present invention is intended to cover modifications and variations.

What is claimed is:

1. A device for improving the function of a heart, the device comprising:
an elongate member configured to be positioned transverse a chamber of the heart; and
a release mechanism connected to the elongate member, the release mechanism being configured to releasably engage with each of a plurality of anchoring members having differing configurations to releasably attach the elongate member to each of the plurality of anchoring members one at a time.

2. The device of claim 1, wherein each of the plurality of anchoring members defines a recess configured to receive the release mechanism.

3. The device of claim 2, wherein the recess is configured to receive the release mechanism when the release mechanism is in a first position.

4. The device of claim 3, further comprising a projection defining a portion of the recess, the projection being configured to prevent passage of the release mechanism through each of the plurality of anchoring members when the release mechanism is in the first position.

5. The device of claim 4, wherein the projection defines an opening configured to permit passage of the release mechanism therethrough when the release mechanism is in a second position.

6. The device of claim 2, wherein the release mechanism is in the form of a block.

7. The device of claim 1, wherein the release mechanism is moveable relative to the elongate member.

8. The device of claim 7, wherein the release member is configured to articulate relative to the elongate member.

9. The device of claim 1, wherein the release member is moveable between a first position wherein the release mechanism is releasable from each of the plurality of anchoring members and a second position wherein the release mechanism is engageable with each of the plurality of anchoring members.

10. The device of claim 1, further comprising at least one of the plurality of anchoring members having differing configurations.

11. The device of claim 10, further comprising an additional anchoring member for securing the elongate member relative to the heart, the additional anchoring member being configured to be attached to the elongate member.

12. The device of claim 11, wherein the at least one anchoring member is configured to be connected to the elongate member at a first end of the elongate member and the additional anchoring member is configured to be attached to the elongate member at a second end of the elongate member.

13. The device of claim 11, wherein the at least one anchoring member is configured to be positioned on a posterior side of a mitral valve and wherein the additional anchoring member is configured to be positioned on an anterior side of the mitral valve.

14. The device of claim 13, wherein each of the at least one anchoring member and the additional anchoring member is configured to be positioned on an exterior surface of a wall of the heart.

15. The device of claim 10, wherein the at least one anchoring member comprises a first contact zone and a second contact zone, the first and second contact zones being configured to rest on an exterior surface of a wall of the heart.

16. The device of claim 15, wherein the at least one anchoring member further comprises a bridge connecting the first contact zone and the second contact zone.

17. The device of claim 15, wherein the first contact zone is configured to be positioned adjacent an annulus of a mitral valve and wherein the second contact zone is configured to be positioned approximately at a level of papillary muscles of the mitral valve.

18. The device of claim 17, wherein the first contact zone is configured to be positioned on a posterior side of the annulus.

19. The device of claim 16, wherein the bridge defines a recess configured to receive the release mechanism.

20. The device of claim 10, further comprising a covering on at least a portion of the at least one anchoring member.

21. The device of claim 20, wherein the covering is configured to facilitate tissue ingrowth.

22. The device of claim 1, wherein the plurality of differing anchoring members include anchoring members having differing sizes and/or shapes.

23. The device of claim 1, wherein the device is configured to improve valve function.

24. The device of claim 1, wherein the chamber is a left ventricle.

25. A method for improving the function of a heart, the method comprising:
providing a plurality of anchoring members having differing configurations;
providing an elongate member and a release mechanism connected to the elongate member, the release mechanism being configured to releasably engage with each of the plurality of anchoring members;
selecting one of the plurality of anchoring members;
positioning the elongate member transverse a chamber of the heart; and
engaging the release mechanism with the selected anchoring member so as to releasably attach the elongate member to the selected anchoring member.

26. A method of delivering a device to be positioned relative to a heart chamber, the method comprising:
providing an elongate member having a first end and a second end, the second end having an expandable anchoring member attached thereto;
advancing the first end of the elongate member through a first heart wall, a septal wall, and a second heart wall substantially opposite the septal wall such that the elongate member extends substantially transverse a heart chamber; and
expanding the expandable anchoring member such that the expandable anchoring member prevents the second end of the elongate member from being able to pass through the septal wall and into the heart chamber.

27. The method of claim 26, wherein the expanding the expandable anchoring member includes inflating the expandable anchoring member.

28. The method of claim 27, wherein the inflating the expandable anchoring member includes filling the expandable anchoring member with a curable substance.

29. The method of claim 28, further comprising connecting an injection mechanism to the expandable anchoring member to fill the expandable anchoring member with a curable substance.

30. The method of claim 26, wherein the expandable anchoring member is a self-expandable anchoring member and expanding the self-expandable anchoring member includes allowing the anchoring member to self-expand.

31. The method of claim 30, wherein allowing the anchoring member to self-expand includes removing the expandable anchor from a housing containing the expandable anchor.

32. The method of claim 31, wherein the housing is defined by a delivery probe.

33. The method of claim 26, wherein the advancing the elongate member includes advancing the elongate member until the expandable anchor is in a heart chamber surrounded by the first wall and the septal wall.

34. The method of claim 33, wherein the advancing the elongate member includes advancing the elongate member with the expandable anchor in a collapsed configuration.

35. The method of claim 26, further comprising attaching an additional anchoring member to the first end of the elongate member.

36. The method of claim 35, wherein the attaching the additional anchoring member includes attaching the additional anchoring member such that the elongate member is in tension.

37. The method of claim 26, wherein the heart chamber is a left ventricle.

38. A device for securing an elongate member in a position transverse at least one heart chamber, the device comprising:
an anchor assembly configured to be secured to the elongate member, the anchor assembly having a collapsed configuration and an expanded configuration and comprising
a first arm,
a second arm, and
at least one biasing member connecting the first arm and the second arm,
wherein, in the absence of external force, the biasing member is configured to exert a biasing force on the first arm and the second arm such that the anchor assembly is in the expanded configuration.

39. The device of claim 38, wherein the at least one biasing member includes two biasing members.

40. The device of claim 39, wherein one of the biasing members connects first end portions of the first arm and the second arm to each other and the other of the biasing members connects second end portions of the first arm and the second arm to each other.

41. The device of claim 38, wherein the first arm and the second arm are substantially perpendicular to each other when the anchor assembly is in the expanded configuration.

42. The device of claim 38, wherein the first arm and the second arm are substantially parallel to each other when the anchor assembly is in the collapsed configuration.

43. The device of claim 38, wherein the first arm is configured to nest in the second arm when the anchor assembly is in the collapsed configuration.

44. The device of claim 43, wherein the first arm has a substantially circular cross-section and the second arm has a substantially parabolic cross-section.

45. The device of claim 38, wherein the at least one biasing member is a spring.

46. The device of claim 38, wherein the biasing member is made of spring tempered stainless steel.

47. The device of claim 38, wherein the first arm and the second arm are pivotable relative to each other.

48. The device of claim 47, wherein the first arm and the second arm are pivotably connected via the elongate member.

49. An alignment device comprising:
an arm; and
a tissue engaging member configured to engage a tissue surface connected to the arm, the tissue engaging member comprising
a cover defining a cover opening, and
a rotatable insert defining a plurality of openings configured to be individually aligned with the cover opening by rotating the insert with respect to the cover,
wherein, when the cover opening and one of the plurality of openings are aligned, the cover opening and one of the plurality of openings are configured to receive a needle assembly.

50. The alignment device of claim 49, wherein the tissue engaging member further comprises a vacuum chamber.

51. The alignment device of claim 49, wherein a first sloped surface leads to the cover opening and second sloped surfaces lead to the plurality of openings.

52. The alignment device of claim 49, further comprising a plate, the plate defining a plurality of receiving portions configured to align with the plurality of openings in the rotatable insert.

53. The alignment device of claim 52, wherein the rotatable insert is disposed between the plate and the cover.

54. The alignment device of claim 52, wherein each of the plurality of receiving portions is configured to capture the needle assembly once a tip portion of the needle assembly has been advanced into the receiving portion.

55. The alignment device of claim 54, wherein each of the plurality of receiving portions defines at least one opening configured to allow passage of the needle assembly in a first direction.

56. The alignment device of claim 55, wherein each of the plurality of receiving portions further includes at least one tab configured to engage a detent in the needle assembly.

57. The alignment device of claim 56, wherein the at least one tab is configured to prevent the needle assembly from passage through the at least one opening in a second direction opposite to the first direction.
